SEQUENCE LISTING (666 sequences)

SEQ ID NO: 1 - protein, 6 aa, Artificial Sequence
  Synthetic peptide
  misc_feature (2)..(3): Xaa can be any naturally occurring amino acid
CXXCST

SEQ ID NO: 2 - protein, 6 aa, Artificial Sequence
  synthetic peptide
  misc_feature (4)..(5): Xaa can be any naturally occurring amino acid
CSTXXC

SEQ ID NO: 3 - protein, 9 aa, Artificial Sequence
  sequence listing
LALEGSLQK

SEQ ID NO: 4 - protein, 19 aa, Artificial Sequence
  synhtetic peptide
  misc_feature (2)..(3): Xaa can be any naturally occurring amino acid
CXXCSTSLQPLALEGSLQK

SEQ ID NO: 5 - protein, 19 aa, Artificial Sequence
  synthetic peptide
  misc_feature (4)..(5): Xaa can be any naturally occurring amino acid
CSTXXCSLQPLALEGSLQK

SEQ ID NO: 6 - protein, 17 aa, Artificial Sequence
  synthetic peptide
  misc_feature (2)..(3): Xaa can be any naturally occurring amino acid
CXXCSLQPLALEGSLQK

SEQ ID NO: 7 - protein, 20 aa, Artificial Sequence
  synthetic peptide
  misc_feature (3)..(4): Xaa can be any naturally occurring amino acid
HCXXCSTSLQPLALEGSLQK

SEQ ID NO: 8 - protein, 20 aa, Artificial Sequence
  synthetic peptide
  misc_feature (5)..(6): Xaa can be any naturally occurring amino acid
HCSTXXCSLQPLALEGSLQK

SEQ ID NO: 9 - protein, 18 aa, Artificial Sequence
  synthetic peptide
  misc_feature (3)..(4): Xaa can be any naturally occurring amino acid
HCXXCSLQPLALEGSLQK

SEQ ID NO: 10 - protein, 21 aa, Artificial Sequence
  synthetic peptide
  misc_feature (2)..(3): Xaa can be any naturally occurring amino acid
CXXCSTSLQPLALEGSLQKRG

SEQ ID NO: 11 - protein, 21 aa, Artificial Sequence
  synthetic peptide
  misc_feature (4)..(5): Xaa can be any naturally occurring amino acid
CSTXXCSLQPLALEGSLQKRG

SEQ ID NO: 12 - protein, 19 aa, Artificial Sequence
  synthetic peptide
  misc_feature (2)..(3): Xaa can be any naturally occurring amino acid
CXXCSLQPLALEGSLQKRG

SEQ ID NO: 13 - protein, 22 aa, Artificial Sequence
  synthetic peptide
  misc_feature (3)..(4): Xaa can be any naturally occurring amino acid
HCXXCSTSLQPLALEGSLQKRG

SEQ ID NO: 14 - protein, 22 aa, Artificial Sequence
  synthetic peptide
  misc_feature (5)..(6): Xaa can be any naturally occurring amino acid
HCSTXXCSLQPLALEGSLQKRG

SEQ ID NO: 15 - protein, 20 aa, Artificial Sequence
  synthetic peptide
  misc_feature (3)..(4): Xaa can be any naturally occurring amino acid
HCXXCSLQPLALEGSLQKRG

SEQ ID NO: 16 - protein, 6 aa, Artificial Sequence
  synthetic peptide
CPYCST

SEQ ID NO: 17 - protein, 6 aa, Artificial Sequence
  synthetic peptide
CSTPYC

SEQ ID NO: 18 - protein, 4 aa, Artificial Sequence
  synthetic peptide
  misc_feature (2)..(3): Xaa can be any naturally occurring amino acid
CXXC

SEQ ID NO: 19 - protein, 4 aa, Artificial Sequence
  synthetic peptide
CPYC

SEQ ID NO: 20 - protein, 15 aa, Artificial Sequence
  synthetic peptide
SLQPLALEGSLQKRG

SEQ ID NO: 21 - protein, 4 aa, Artificial Sequence
  synthetic peptide
  misc_feature (2)..(3): Xaa can be any naturally occurring amino acid
SXXC

SEQ ID NO: 22 - protein, 4 aa, Artificial Sequence
  synthetic peptide
  misc_feature (2)..(3): Xaa can be any naturally occurring amino acid
TXXC

SEQ ID NO: 23 - protein, 4 aa, Artificial Sequence
  synthetic peptide
  misc_feature (2)..(3): Xaa can be any naturally occurring amino acid
CXXS

SEQ ID NO: 24 - protein, 4 aa, Artificial Sequence
  synthetic peptide
  misc_feature (2)..(3): Xaa can be any naturally occurring amino acid
CXXT

SEQ ID NO: 25 - protein, 22 aa, Artificial Sequence
  synthetic peptide
HCPYCVRSLQPLALEGSLQKRG

SEQ ID NO: 26 - protein, 20 aa, Artificial Sequence
  synthetic peptide
HCPYCSLQPLALEGSLQKRG

SEQ ID NO: 27 - protein, 2 aa, Artificial Sequence
  synthetic peptide
CC

SEQ ID NO: 28 - protein, 3 aa, Artificial Sequence
  synthetic peptide
  misc_feature (2)..(2): Xaa can be any naturally occurring amino acid
CXC

SEQ ID NO: 29 - protein, 5 aa, Artificial Sequence
  synthetic peptide
  misc_feature (2)..(4): Xaa can be any naturally occurring amino acid
CXXXC

SEQ ID NO: 30 - protein, 6 aa, Artificial Sequence
  synthetic peptide
  misc_feature (2)..(5): Xaa can be any naturally occurring amino acid
CXXXXC

SEQ ID NO: 31 - protein, 7 aa, Artificial Sequence
  synthetic peptide
  misc_feature (2)..(6): Xaa can be any naturally occurring amino acid
CXXXXXC

SEQ ID NO: 32 - protein, 8 aa, Artificial Sequence
  synthetic peptide
  misc_feature (2)..(7): Xaa can be any naturally occurring amino acid
CXXXXXXC

SEQ ID NO: 33 - protein, 4 aa, Artificial Sequence
  synthetic peptide
  PEPTIDE (1)..(1): C, S, or T
  misc_feature (2)..(3): Xaa can be any naturally occurring amino acid
XXXC

SEQ ID NO: 34 - protein, 5 aa, Artificial Sequence
  synthetic peptide
  PEPTIDE (1)..(1): C, S, or T
  misc_feature (2)..(4): Xaa can be any naturally occurring amino acid
XXXXC

SEQ ID NO: 35 - protein, 6 aa, Artificial Sequence
  synthetic peptide
  PEPTIDE (1)..(1): C, S, or T
  misc_feature (2)..(5): Xaa can be any naturally occurring amino acid
XXXXXC

SEQ ID NO: 36 - protein, 7 aa, Artificial Sequence
  synthetic peptide
  PEPTIDE (1)..(1): C, S, or T
  misc_feature (2)..(6): Xaa can be any naturally occurring amino acid
XXXXXXC

SEQ ID NO: 37 - protein, 8 aa, Artificial Sequence
  synthetic peptide
  PEPTIDE (1)..(1): C, S, or T
  misc_feature (2)..(7): Xaa can be any naturally occurring amino acid
XXXXXXXC

SEQ ID NO: 38 - protein, 4 aa, Artificial Sequence
  synthetic peptide
  misc_feature (1)..(1): Xaa can be any naturally occurring amino acid
  PEPTIDE (2)..(2): C, S, or T
  misc_feature (3)..(3): Xaa can be any naturally occurring amino acid
XXXC

SEQ ID NO: 39 - protein, 5 aa, Artificial Sequence
  synthetic peptide
  misc_feature (1)..(1): Xaa can be any naturally occurring amino acid
  PEPTIDE (2)..(2): C, S, or T
  misc_feature (3)..(4): Xaa can be any naturally occurring amino acid
XXXXC

SEQ ID NO: 40 - protein, 6 aa, Artificial Sequence
  synthetic peptide
  misc_feature (1)..(1): Xaa can be any naturally occurring amino acid
  PEPTIDE (2)..(2): C, S, or T
  misc_feature (3)..(5): Xaa can be any naturally occurring amino acid
XXXXXC

SEQ ID NO: 41 - protein, 7 aa, Artificial Sequence
  synthetic peptide
  misc_feature (1)..(1): Xaa can be any naturally occurring amino acid
  PEPTIDE (2)..(2): C, S, or T
  misc_feature (3)..(6): Xaa can be any naturally occurring amino acid
XXXXXXC

SEQ ID NO: 42 - protein, 8 aa, Artificial Sequence
  synthetic peptide
  misc_feature (1)..(1): Xaa can be any naturally occurring amino acid
  PEPTIDE (2)..(2): C, S, or T
  misc_feature (3)..(7): Xaa can be any naturally occurring amino acid
XXXXXXXC

SEQ ID NO: 43 - protein, 9 aa, Artificial Sequence
  synthetic peptide
  misc_feature (1)..(1): Xaa can be any naturally occurring amino acid
  PEPTIDE (2)..(2): C, S, or T
  misc_feature (3)..(8): Xaa can be any naturally occurring amino acid
XXXXXXXXC

SEQ ID NO: 44 - protein, 4 aa, Artificial Sequence
  synthetic peptide
  misc_feature (1)..(2): Xaa can be any naturally occurring amino acid
  PEPTIDE (3)..(3): C, S, or T
XXXC

SEQ ID NO: 45 - protein, 5 aa, Artificial Sequence
  synthetic peptide
  misc_feature (1)..(2): Xaa can be any naturally occurring amino acid
  PEPTIDE (3)..(3): C, S, or T
  misc_feature (4)..(4): Xaa can be any naturally occurring amino acid
XXXXC

SEQ ID NO: 46 - protein, 6 aa, Artificial Sequence
  synthetic peptide
  misc_feature (1)..(2): Xaa can be any naturally occurring amino acid
  PEPTIDE (3)..(3): C, S, or T
  misc_feature (4)..(5): Xaa can be any naturally occurring amino acid
XXXXXC

SEQ ID NO: 47 - protein, 7 aa, Artificial Sequence
  synthetic peptide
  misc_feature (1)..(2): Xaa can be any naturally occurring amino acid
  PEPTIDE (3)..(3): C, S, or T
  misc_feature (4)..(6): Xaa can be any naturally occurring amino acid
XXXXXXC

SEQ ID NO: 48 - protein, 8 aa, Artificial Sequence
  synthetic peptide
  misc_feature (1)..(2): Xaa can be any naturally occurring amino acid
  PEPTIDE (3)..(3): C, S, or T
  misc_feature (4)..(7): Xaa can be any naturally occurring amino acid
XXXXXXXC

SEQ ID NO: 49 - protein, 9 aa, Artificial Sequence
  synthetic peptide
  misc_feature (1)..(2): Xaa can be any naturally occurring amino acid
  PEPTIDE (3)..(3): C, S, or T
  misc_feature (4)..(8): Xaa can be any naturally occurring amino acid
XXXXXXXXC

SEQ ID NO: 50 - protein, 10 aa, Artificial Sequence
  synthetic peptide
  misc_feature (1)..(2): Xaa can be any naturally occurring amino acid
  PEPTIDE (3)..(3): C, S, or T
  misc_feature (4)..(9): Xaa can be any naturally occurring amino acid
XXXXXXXXXC

SEQ ID NO: 51 - protein, 4 aa, Artificial Sequence
  synthetic peptide
  misc_feature (2)..(3): Xaa can be any naturally occurring amino acid
  PEPTIDE (4)..(4): C, S, or T
CXXX

SEQ ID NO: 52 - protein, 5 aa, Artificial Sequence
  synthetic peptide
  misc_feature (2)..(4): Xaa can be any naturally occurring amino acid
  PEPTIDE (5)..(5): C, S, or T
CXXXX

SEQ ID NO: 53 - protein, 6 aa, Artificial Sequence
  synthetic peptide
  misc_feature (2)..(5): Xaa can be any naturally occurring amino acid
  PEPTIDE (6)..(6): C, S, or T
CXXXXX

SEQ ID NO: 54 - protein, 7 aa, Artificial Sequence
  synthetic peptide
  misc_feature (2)..(6): Xaa can be any naturally occurring amino acid
  PEPTIDE (7)..(7): C, S, or T
CXXXXXX

SEQ ID NO: 55 - protein, 8 aa, Artificial Sequence
  synthetic peptide
  misc_feature (2)..(7): Xaa can be any naturally occurring amino acid
  PEPTIDE (8)..(8): C, S, or T
CXXXXXXX

SEQ ID NO: 56 - protein, 4 aa, Artificial Sequence
  synthetic peptide
  misc_feature (1)..(1): Xaa can be any naturally occurring amino acid
  misc_feature (3)..(3): Xaa can be any naturally occurring amino acid
  PEPTIDE (4)..(4): C, S, or T
XCXX

SEQ ID NO: 57 - protein, 5 aa, Artificial Sequence
  synthetic peptide
  misc_feature (1)..(1): Xaa can be any naturally occurring amino acid
  misc_feature (3)..(4): Xaa can be any naturally occurring amino acid
  PEPTIDE (5)..(5): C, S, or T
XCXXX

SEQ ID NO: 58 - protein, 6 aa, Artificial Sequence
  synthetic peptide
  misc_feature (1)..(1): Xaa can be any naturally occurring amino acid
  misc_feature (3)..(5): Xaa can be any naturally occurring amino acid
  PEPTIDE (6)..(6): C, S, or T
XCXXXX

SEQ ID NO: 59 - protein, 7 aa, Artificial Sequence
  synthetic peptide
  misc_feature (1)..(1): Xaa can be any naturally occurring amino acid
  misc_feature (3)..(6): Xaa can be any naturally occurring amino acid
  PEPTIDE (7)..(7): C, S, or T
XCXXXXX

SEQ ID NO: 60 - protein, 8 aa, Artificial Sequence
  synthetic peptide
  misc_feature (1)..(1): Xaa can be any naturally occurring amino acid
  misc_feature (3)..(7): Xaa can be any naturally occurring amino acid
  PEPTIDE (8)..(8): C, S, or T
XCXXXXXX

SEQ ID NO: 61 - protein, 9 aa, Artificial Sequence
  synthetic peptide
  misc_feature (1)..(1): Xaa can be any naturally occurring amino acid
  misc_feature (3)..(8): Xaa can be any naturally occurring amino acid
  PEPTIDE (9)..(9): C, S, or T
XCXXXXXXX

SEQ ID NO: 62 - protein, 4 aa, Artificial Sequence
  synthetic peptide
  misc_feature (1)..(2): Xaa can be any naturally occurring amino acid
  PEPTIDE (4)..(4): C, S, or T
XXCX

SEQ ID NO: 63 - protein, 5 aa, Artificial Sequence
  synthetic peptide
  misc_feature (1)..(2): Xaa can be any naturally occurring amino acid
  misc_feature (4)..(4): Xaa can be any naturally occurring amino acid
  PEPTIDE (5)..(5): C, S, or T
XXCXX

SEQ ID NO: 64 - protein, 6 aa, Artificial Sequence
  synthetic peptide
  misc_feature (1)..(2): Xaa can be any naturally occurring amino acid
  misc_feature (4)..(5): Xaa can be any naturally occurring amino acid
  PEPTIDE (6)..(6): C, S, or T
XXCXXX

SEQ ID NO: 65 - protein, 7 aa, Artificial Sequence
  synthetic peptide
  misc_feature (1)..(2): Xaa can be any naturally occurring amino acid
  misc_feature (4)..(6): Xaa can be any naturally occurring amino acid
  PEPTIDE (7)..(7): C, S, or T
XXCXXXX

SEQ ID NO: 66 - protein, 8 aa, Artificial Sequence
  synthetic peptide
  misc_feature (1)..(2): Xaa can be any naturally occurring amino acid
  misc_feature (4)..(7): Xaa can be any naturally occurring amino acid
  PEPTIDE (8)..(8): C, S, or T
XXCXXXXX

SEQ ID NO: 67 - protein, 9 aa, Artificial Sequence
  synthetic peptide
  misc_feature (1)..(2): Xaa can be any naturally occurring amino acid
  misc_feature (4)..(8): Xaa can be any naturally occurring amino acid
  PEPTIDE (9)..(9): C, S, or T
XXCXXXXXX

SEQ ID NO: 68 - protein, 10 aa, Artificial Sequence
  synthetic peptide
  misc_feature (1)..(2): Xaa can be any naturally occurring amino acid
  misc_feature (4)..(9): Xaa can be any naturally occurring amino acid
  PEPTIDE (10)..(10): C, S, or T
XXCXXXXXXX

SEQ ID NO: 69 - protein, 5 aa, Artificial Sequence
  synthetic peptide
  misc_feature (3)..(4): Xaa can be any naturally occurring amino acid
HCXXC

SEQ ID NO: 70 - protein, 5 aa, Artificial Sequence
  synthetic peptide
  misc_feature (3)..(4): Xaa can be any naturally occurring amino acid
KCXXC

SEQ ID NO: 71 - protein, 5 aa, Artificial Sequence
  synthetic peptide
  misc_feature (3)..(4): Xaa can be any naturally occurring amino acid
RCXXC

SEQ ID NO: 72 - protein, 6 aa, Artificial Sequence
  synhtetic peptifde
  misc_feature (4)..(5): Xaa can be any naturally occurring amino acid
KHCXXC

SEQ ID NO: 73 - protein, 6 aa, Artificial Sequence
  synthetic peptide
  misc_feature (4)..(5): Xaa can be any naturally occurring amino acid
HKCXXC

SEQ ID NO: 74 - protein, 6 aa, Artificial Sequence
  synthetic peptide
  misc_feature (4)..(5): Xaa can be any naturally occurring amino acid
RHCXXC

SEQ ID NO: 75 - protein, 6 aa, Artificial Sequence
  synthetic peptide
  misc_feature (4)..(5): Xaa can be any naturally occurring amino acid
HRCXXC

SEQ ID NO: 76 - protein, 6 aa, Artificial Sequence
  synthetic peptide
  misc_feature (4)..(5): Xaa can be any naturally occurring amino acid
KRCXXC

SEQ ID NO: 77 - protein, 6 aa, Artificial Sequence
  synthetic peptide
  misc_feature (4)..(5): Xaa can be any naturally occurring amino acid
RKCXXC

SEQ ID NO: 78 - protein, 4 aa, Artificial Sequence
  synthetic peptide
  MISC_FEATURE (1)..(1): Xaa = basic amino acid
  MISC_FEATURE (3)..(3): Xaa = any amino acid
XCXC

SEQ ID NO: 79 - protein, 5 aa, Artificial Sequence
  synthetic peptide
  MISC_FEATURE (1)..(1): Xaa = basic amino acid
  MISC_FEATURE (3)..(4): Xaa = any amino acid
XCXXC

SEQ ID NO: 80 - protein, 6 aa, Artificial Sequence
  synthetic peptide
  MISC_FEATURE (1)..(1): Xaa = basic amino acid
  MISC_FEATURE (3)..(5): Xaa = any amino acid
XCXXXC

SEQ ID NO: 81 - protein, 4 aa, Artificial Sequence
  synthetic peptide
  MISC_FEATURE (1)..(2): Xaa = basic amino acid
XXCC

SEQ ID NO: 82 - protein, 5 aa, Artificial Sequence
  synthetic peptide
  MISC_FEATURE (1)..(2): Xaa = basic amino acid
  MISC_FEATURE (4)..(4): Xaa = any amino acid
XXCXC

SEQ ID NO: 83 - protein, 6 aa, Artificial Sequence
  synthetic peptide
  MISC_FEATURE (1)..(2): Xaa = basic amino acid
  MISC_FEATURE (4)..(5): Xaa = any amino acid
XXCXXC

SEQ ID NO: 84 - protein, 7 aa, Artificial Sequence
  synthetic peptide
  MISC_FEATURE (1)..(2): Xaa = basic amino acid
  MISC_FEATURE (4)..(6): Xaa = any amino acid
XXCXXXC

SEQ ID NO: 85 - protein, 4 aa, Artificial Sequence
  synthetic peptide
  misc_feature (3)..(3): Xaa can be any naturally occurring amino acid
HCXC

SEQ ID NO: 86 - protein, 4 aa, Artificial Sequence
  synthetic peptide
  misc_feature (3)..(3): Xaa can be any naturally occurring amino acid
KCXC

SEQ ID NO: 87 - protein, 4 aa, Artificial Sequence
  synthetic peptide
  misc_feature (3)..(3): Xaa can be any naturally occurring amino acid
RCXC

SEQ ID NO: 88 - protein, 5 aa, Artificial Sequence
  synthetic peptide
  misc_feature (4)..(4): Xaa can be any naturally occurring amino acid
KHCXC

SEQ ID NO: 89 - protein, 5 aa, Artificial Sequence
  synthetic peptide
  misc_feature (4)..(4): Xaa can be any naturally occurring amino acid
HKCXC

SEQ ID NO: 90 - protein, 5 aa, Artificial Sequence
  synthetic peptide
  misc_feature (4)..(4): Xaa can be any naturally occurring amino acid
RHCXC

SEQ ID NO: 91 - protein, 5 aa, Artificial Sequence
  synthetic peptide
  misc_feature (4)..(4): Xaa can be any naturally occurring amino acid
RHCXC

SEQ ID NO: 92 - protein, 5 aa, Artificial Sequence
  synthetic peptide
  misc_feature (4)..(4): Xaa can be any naturally occurring amino acid
RKCXC

SEQ ID NO: 93 - protein, 5 aa, Artificial Sequence
  synthetic peptide
  misc_feature (4)..(4): Xaa can be any naturally occurring amino acid
KRCXC

SEQ ID NO: 94 - protein, 6 aa, Artificial Sequence
  synthetic peptide
  misc_feature (3)..(5): Xaa can be any naturally occurring amino acid
HCXXXC

SEQ ID NO: 95 - protein, 6 aa, Artificial Sequence
  synthetic peptide
  misc_feature (3)..(5): Xaa can be any naturally occurring amino acid
KCXXXC

SEQ ID NO: 96 - protein, 6 aa, Artificial Sequence
  synthetic peptide
  misc_feature (3)..(5): Xaa can be any naturally occurring amino acid
RCXXXC

SEQ ID NO: 97 - protein, 7 aa, Artificial Sequence
  synthetic peptide
  misc_feature (4)..(6): Xaa can be any naturally occurring amino acid
KHCXXXC

SEQ ID NO: 98 - protein, 7 aa, Artificial Sequence
  synthetic peptide
  misc_feature (4)..(6): Xaa can be any naturally occurring amino acid
HKCXXXC

SEQ ID NO: 99 - protein, 7 aa, Artificial Sequence
  synthetic peptide
  misc_feature (4)..(6): Xaa can be any naturally occurring amino acid
RHCXXXC

SEQ ID NO: 100 - protein, 7 aa, Artificial Sequence
  synthetic peptide
  misc_feature (4)..(6): Xaa can be any naturally occurring amino acid
HRCXXXC

SEQ ID NO: 101 - protein, 7 aa, Artificial Sequence
  synthetic peptide
  misc_feature (4)..(6): Xaa can be any naturally occurring amino acid
RKCXXXC

SEQ ID NO: 102 - protein, 7 aa, Artificial Sequence
  synthetic peptide
  misc_feature (4)..(6): Xaa can be any naturally occurring amino acid
KRCXXXC

SEQ ID NO: 103 - protein, 6 aa, Artificial Sequence
  synthetic peptide
  misc_feature (3)..(5): Xaa can be any naturally occurring amino acid
HCXXXC

SEQ ID NO: 104 - protein, 6 aa, Artificial Sequence
  synthetic peptide
  misc_feature (3)..(5): Xaa can be any naturally occurring amino acid
KCXXXC

SEQ ID NO: 105 - protein, 6 aa, Artificial Sequence
  synthetic peptide
  misc_feature (3)..(5): Xaa can be any naturally occurring amino acid
RCXXXC

SEQ ID NO: 106 - protein, 7 aa, Artificial Sequence
  synthetic peptide
  misc_feature (4)..(6): Xaa can be any naturally occurring amino acid
KHCXXXC

SEQ ID NO: 107 - protein, 7 aa, Artificial Sequence
  synthetic peptide
  misc_feature (4)..(6): Xaa can be any naturally occurring amino acid
HKCXXXC

SEQ ID NO: 108 - protein, 7 aa, Artificial Sequence
  synthetic peptide
  misc_feature (4)..(6): Xaa can be any naturally occurring amino acid
RHCXXXC

SEQ ID NO: 109 - protein, 7 aa, Artificial Sequence
  synthetic peptide
  misc_feature (4)..(6): Xaa can be any naturally occurring amino acid
HRCXXXC

SEQ ID NO: 110 - protein, 7 aa, Artificial Sequence
  synthetic peptide
  misc_feature (4)..(6): Xaa can be any naturally occurring amino acid
RKCXXXC

SEQ ID NO: 111 - protein, 7 aa, Artificial Sequence
  synthetic peptide
  misc_feature (4)..(6): Xaa can be any naturally occurring amino acid
KRCXXXC

SEQ ID NO: 112 - protein, 7 aa, Artificial Sequence
  synthetic peptide
  misc_feature (3)..(6): Xaa can be any naturally occurring amino acid
HCXXXXC

SEQ ID NO: 113 - protein, 7 aa, Artificial Sequence
  synthetic peptide
  misc_feature (3)..(6): Xaa can be any naturally occurring amino acid
KCXXXXC

SEQ ID NO: 114 - protein, 7 aa, Artificial Sequence
  synthetic peptide
  misc_feature (3)..(6): Xaa can be any naturally occurring amino acid
RCXXXXC

SEQ ID NO: 115 - protein, 8 aa, Artificial Sequence
  synthetic peptide
  misc_feature (4)..(7): Xaa can be any naturally occurring amino acid
KHCXXXXC

SEQ ID NO: 116 - protein, 8 aa, Artificial Sequence
  synthetic peptide
  misc_feature (4)..(7): Xaa can be any naturally occurring amino acid
HKCXXXXC

SEQ ID NO: 117 - protein, 8 aa, Artificial Sequence
  synthetic peptide
  misc_feature (4)..(7): Xaa can be any naturally occurring amino acid
RHCXXXXC

SEQ ID NO: 118 - protein, 8 aa, Artificial Sequence
  synthetic peptide
  misc_feature (4)..(7): Xaa can be any naturally occurring amino acid
HRCXXXXC

SEQ ID NO: 119 - protein, 8 aa, Artificial Sequence
  synthetic peptide
  misc_feature (4)..(7): Xaa can be any naturally occurring amino acid
RKCXXXXC

SEQ ID NO: 120 - protein, 8 aa, Artificial Sequence
  synthetic peptide
  misc_feature (4)..(7): Xaa can be any naturally occurring amino acid
KRCXXXXC

SEQ ID NO: 121 - protein, 8 aa, Artificial Sequence
  synthetic peptide
  misc_feature (3)..(7): Xaa can be any naturally occurring amino acid
HCXXXXXC

SEQ ID NO: 122 - protein, 8 aa, Artificial Sequence
  synthetic peptide
  misc_feature (3)..(7): Xaa can be any naturally occurring amino acid
KCXXXXXC

SEQ ID NO: 123 - protein, 8 aa, Artificial Sequence
  synthetic peptide
  misc_feature (3)..(7): Xaa can be any naturally occurring amino acid
RCXXXXXC

SEQ ID NO: 124 - protein, 9 aa, Artificial Sequence
  synthetic peptide
  misc_feature (4)..(8): Xaa can be any naturally occurring amino acid
KHCXXXXXC

SEQ ID NO: 125 - protein, 11 aa, Artificial Sequence
  synthetic peptide
  misc_feature (4)..(10): Xaa can be any naturally occurring amino acid
HKCXXXXXXXC

SEQ ID NO: 126 - protein, 9 aa, Artificial Sequence
  synthetic peptide
  misc_feature (4)..(8): Xaa can be any naturally occurring amino acid
RHCXXXXXC

SEQ ID NO: 127 - protein, 9 aa, Artificial Sequence
  synthetic peptide
  misc_feature (4)..(8): Xaa can be any naturally occurring amino acid
HRCXXXXXC

SEQ ID NO: 128 - protein, 9 aa, Artificial Sequence
  synthetic peptide
  misc_feature (4)..(8): Xaa can be any naturally occurring amino acid
RKCXXXXXC

SEQ ID NO: 129 - protein, 9 aa, Artificial Sequence
  synthetic peptide
  misc_feature (4)..(8): Xaa can be any naturally occurring amino acid
KRCXXXXXC

SEQ ID NO: 130 - protein, 9 aa, Artificial Sequence
  synthetic peptide
  misc_feature (3)..(8): Xaa can be any naturally occurring amino acid
HCXXXXXXC

SEQ ID NO: 131 - protein, 9 aa, Artificial Sequence
  synthetic peptide
  misc_feature (3)..(8): Xaa can be any naturally occurring amino acid
KCXXXXXXC

SEQ ID NO: 132 - protein, 9 aa, Artificial Sequence
  synthetic peptide
  misc_feature (3)..(8): Xaa can be any naturally occurring amino acid
RCXXXXXXC

SEQ ID NO: 133 - protein, 10 aa, Artificial Sequence
  synthetic peptide
  misc_feature (4)..(9): Xaa can be any naturally occurring amino acid
KHCXXXXXXC

SEQ ID NO: 134 - protein, 10 aa, Artificial Sequence
  synthetic peptide
  misc_feature (4)..(9): Xaa can be any naturally occurring amino acid
HKCXXXXXXC

SEQ ID NO: 135 - protein, 10 aa, Artificial Sequence
  synthetic peptide
  misc_feature (4)..(9): Xaa can be any naturally occurring amino acid
RHCXXXXXXC

SEQ ID NO: 136 - protein, 10 aa, Artificial Sequence
  synthetic peptide
  misc_feature (4)..(9): Xaa can be any naturally occurring amino acid
HRCXXXXXXC

SEQ ID NO: 137 - protein, 10 aa, Artificial Sequence
  synthetic peptide
  misc_feature (4)..(9): Xaa can be any naturally occurring amino acid
RKCXXXXXXC

SEQ ID NO: 138 - protein, 10 aa, Artificial Sequence
  synthetic peptide
  misc_feature (4)..(9): Xaa can be any naturally occurring amino acid
KRCXXXXXXC

SEQ ID NO: 139 - protein, 4 aa, Artificial Sequence
  synthetic peptide
KCHC

SEQ ID NO: 140 - protein, 4 aa, Artificial Sequence
  synthetic peptide
KCKC

SEQ ID NO: 141 - protein, 4 aa, Artificial Sequence
  synthetic peptide
KCRC

SEQ ID NO: 142 - protein, 4 aa, Artificial Sequence
  synthetic peptide
KCGC

SEQ ID NO: 143 - protein, 4 aa, Artificial Sequence
  synthetic peptide
KCAC

SEQ ID NO: 144 - protein, 4 aa, Artificial Sequence
  synthetic peptide
KCVC

SEQ ID NO: 145 - protein, 4 aa, Artificial Sequence
  synthetic peptide
KCLC

SEQ ID NO: 146 - protein, 4 aa, Artificial Sequence
  synthetic peptide
KCIC

SEQ ID NO: 147 - protein, 4 aa, Artificial Sequence
  synthetic peptide
KCMC

SEQ ID NO: 148 - protein, 4 aa, Artificial Sequence
  synthetic peptide
KCFC

SEQ ID NO: 149 - protein, 4 aa, Artificial Sequence
  synthetic peptide
KCWC

SEQ ID NO: 150 - protein, 4 aa, Artificial Sequence
  synthetic peptide
KCPC

SEQ ID NO: 151 - protein, 4 aa, Artificial Sequence
  synthetic peptide
KCSC

SEQ ID NO: 152 - protein, 4 aa, Artificial Sequence
  synthetic peptide
KCTC

SEQ ID NO: 153 - protein, 4 aa, Artificial Sequence
  synthetic peptide
KCYC

SEQ ID NO: 154 - protein, 4 aa, Artificial Sequence
  synthetic peptide
KCNC

SEQ ID NO: 155 - protein, 4 aa, Artificial Sequence
  synthetic peptide
KCQC

SEQ ID NO: 156 - protein, 4 aa, Artificial Sequence
  synthetic peptide
KCDC

SEQ ID NO: 157 - protein, 4 aa, Artificial Sequence
  synthetic peptide
KCEC

SEQ ID NO: 158 - protein, 4 aa, Artificial Sequence
  synthetic peptide
HCHC

SEQ ID NO: 159 - protein, 4 aa, Artificial Sequence
  synthetic peptide
HCKC

SEQ ID NO: 160 - protein, 4 aa, Artificial Sequence
  synthetic peptide
HCRC

SEQ ID NO: 161 - protein, 4 aa, Artificial Sequence
  synthetic peptide
HCGC

SEQ ID NO: 162 - protein, 4 aa, Artificial Sequence
  synthetic peptide
HCAC

SEQ ID NO: 163 - protein, 4 aa, Artificial Sequence
  synthetic peptide
HCVC

SEQ ID NO: 164 - protein, 4 aa, Artificial Sequence
  synthetic peptide
HCLC

SEQ ID NO: 165 - protein, 4 aa, Artificial Sequence
  synthetic peptide
HCIC

SEQ ID NO: 166 - protein, 4 aa, Artificial Sequence
  synthetic peptide
HCMC

SEQ ID NO: 167 - protein, 4 aa, Artificial Sequence
  synthetic peptide
HCFC

SEQ ID NO: 168 - protein, 4 aa, Artificial Sequence
  synthetic peptide
HCWC

SEQ ID NO: 169 - protein, 4 aa, Artificial Sequence
  synthetic peptide
HCPC

SEQ ID NO: 170 - protein, 4 aa, Artificial Sequence
  synthetic peptide
HCSC

SEQ ID NO: 171 - protein, 4 aa, Artificial Sequence
  synthetic peptide
HCTC

SEQ ID NO: 172 - protein, 4 aa, Artificial Sequence
  synthetic peptide
HCYC

SEQ ID NO: 173 - protein, 4 aa, Artificial Sequence
  synthetic peptide
HCNC

SEQ ID NO: 174 - protein, 4 aa, Artificial Sequence
  synthetic peptide
HCQC

SEQ ID NO: 175 - protein, 4 aa, Artificial Sequence
  synthetic peptide
HCDC

SEQ ID NO: 176 - protein, 4 aa, Artificial Sequence
  synthetic peptide
HCEC

SEQ ID NO: 177 - protein, 4 aa, Artificial Sequence
  synthetic peptide
RCHC

SEQ ID NO: 178 - protein, 4 aa, Artificial Sequence
  synthetic peptide
RCKC

SEQ ID NO: 179 - protein, 4 aa, Artificial Sequence
  synthetic peptide
RCRC

SEQ ID NO: 180 - protein, 4 aa, Artificial Sequence
  synthetic peptide
RCGC

SEQ ID NO: 181 - protein, 4 aa, Artificial Sequence
  synthetic peptide
RCAC

SEQ ID NO: 182 - protein, 4 aa, Artificial Sequence
  synthetic peptide
RCVC

SEQ ID NO: 183 - protein, 4 aa, Artificial Sequence
  synthetic peptide
RCLC

SEQ ID NO: 184 - protein, 4 aa, Artificial Sequence
  synthetic peptide
RCIC

SEQ ID NO: 185 - protein, 4 aa, Artificial Sequence
  synthetic peptide
RCMC

SEQ ID NO: 186 - protein, 4 aa, Artificial Sequence
  synthetic peptide
RCFC

SEQ ID NO: 187 - protein, 4 aa, Artificial Sequence
  synthetic peptide
RCWC

SEQ ID NO: 188 - protein, 4 aa, Artificial Sequence
  synthetic peptide
RCPC

SEQ ID NO: 189 - protein, 4 aa, Artificial Sequence
  synthetic peptide
RCSC

SEQ ID NO: 190 - protein, 4 aa, Artificial Sequence
  synthetic peptide
RCTC

SEQ ID NO: 191 - protein, 4 aa, Artificial Sequence
  synthetic peptide
RCYC

SEQ ID NO: 192 - protein, 4 aa, Artificial Sequence
  synthetic peptide
RCNC

SEQ ID NO: 193 - protein, 4 aa, Artificial Sequence
  synthetic peptide
RCQC

SEQ ID NO: 194 - protein, 4 aa, Artificial Sequence
  synthetic peptide
RCDC

SEQ ID NO: 195 - protein, 4 aa, Artificial Sequence
  synthetic peptide
RCEC

SEQ ID NO: 196 - protein, 5 aa, Artificial Sequence
  synthetic peptide
  misc_feature (2)..(2): Xaa can be any naturally occurring amino acid
CXPYC

SEQ ID NO: 197 - protein, 5 aa, Artificial Sequence
  synthetic peptide
  misc_feature (3)..(3): Xaa can be any naturally occurring amino acid
CPXYC

SEQ ID NO: 198 - protein, 5 aa, Artificial Sequence
  synthetic peptide
  misc_feature (4)..(4): Xaa can be any naturally occurring amino acid
CPYXC

SEQ ID NO: 199 - protein, 5 aa, Artificial Sequence
  synthetic peptide
CKPYC

SEQ ID NO: 200 - protein, 5 aa, Artificial Sequence
  synthetic peptide
CRPYC

SEQ ID NO: 201 - protein, 5 aa, Artificial Sequence
  synthetic peptide
CHPYC

SEQ ID NO: 202 - protein, 5 aa, Artificial Sequence
  synthetic peptide
CGPYC

SEQ ID NO: 203 - protein, 5 aa, Artificial Sequence
  synthetic peptide
CAPYC

SEQ ID NO: 204 - protein, 5 aa, Artificial Sequence
  synthetic peptide
CVPYC

SEQ ID NO: 205 - protein, 5 aa, Artificial Sequence
  synthetic peptide
CLPYC

SEQ ID NO: 206 - protein, 5 aa, Artificial Sequence
  synthetic peptide
CIPYC

SEQ ID NO: 207 - protein, 5 aa, Artificial Sequence
  synthetic peptide
CMPYC

SEQ ID NO: 208 - protein, 5 aa, Artificial Sequence
  synthetic peptide
CFPYC

SEQ ID NO: 209 - protein, 5 aa, Artificial Sequence
  synthetic peptide
CWPYC

SEQ ID NO: 210 - protein, 5 aa, Artificial Sequence
  synthetic peptide
CPPYC

SEQ ID NO: 211 - protein, 5 aa, Artificial Sequence
  synthetic peptide
CSPYC

SEQ ID NO: 212 - protein, 5 aa, Artificial Sequence
  synthetic peptide
CTPYC

SEQ ID NO: 213 - protein, 5 aa, Artificial Sequence
  synthetic peptide
CCPYC

SEQ ID NO: 214 - protein, 5 aa, Artificial Sequence
  synthetic peptide
CYPYC

SEQ ID NO: 215 - protein, 5 aa, Artificial Sequence
  synthetic peptide
CNPYC

SEQ ID NO: 216 - protein, 5 aa, Artificial Sequence
  synthetic peptide
CQPYC

SEQ ID NO: 217 - protein, 5 aa, Artificial Sequence
  synthetic peptide
CDPYC

SEQ ID NO: 218 - protein, 5 aa, Artificial Sequence
  synthetic peptide
CEPYC

SEQ ID NO: 219 - protein, 5 aa, Artificial Sequence
  synthetic peptide
CPKYC

SEQ ID NO: 220 - protein, 5 aa, Artificial Sequence
  synthetic peptide
CPRYC

SEQ ID NO: 221 - protein, 5 aa, Artificial Sequence
  synthetic peptide
CPHYC

SEQ ID NO: 222 - protein, 5 aa, Artificial Sequence
  synthetic peptide
CPGYC

SEQ ID NO: 223 - protein, 5 aa, Artificial Sequence
  synthetic peptide
CPAYC

SEQ ID NO: 224 - protein, 5 aa, Artificial Sequence
  synthetic peptide
CPVYC

SEQ ID NO: 225 - protein, 5 aa, Artificial Sequence
  synthetic peptide
CPLYC

SEQ ID NO: 226 - protein, 5 aa, Artificial Sequence
  synthetic peptide
CPIYC

SEQ ID NO: 227 - protein, 5 aa, Artificial Sequence
  synthetic peptide
CPMYC

SEQ ID NO: 228 - protein, 5 aa, Artificial Sequence
  synthetic peptide
CPFYC

SEQ ID NO: 229 - protein, 5 aa, Artificial Sequence
  synthetic peptide
CPWYC

SEQ ID NO: 230 - protein, 5 aa, Artificial Sequence
  synthetic peptide
CPPYC

SEQ ID NO: 231 - protein, 5 aa, Artificial Sequence
  synthetic peptide
CPSYC

SEQ ID NO: 232 - protein, 5 aa, Artificial Sequence
  synthetic peptide
CPTYC

SEQ ID NO: 233 - protein, 5 aa, Artificial Sequence
  synthetic peptide
CPCYC

SEQ ID NO: 234 - protein, 5 aa, Artificial Sequence
  synthetic peptide
CPYYC

SEQ ID NO: 235 - protein, 5 aa, Artificial Sequence
  synthetic peptide
CPNYC

SEQ ID NO: 236 - protein, 5 aa, Artificial Sequence
  synthetic peptide
CPQYC

SEQ ID NO: 237 - protein, 5 aa, Artificial Sequence
  synthetic peptide
CPDYC

SEQ ID NO: 238 - protein, 5 aa, Artificial Sequence
  synthetic peptide
CPEYC

SEQ ID NO: 239 - protein, 5 aa, Artificial Sequence
  synthetic peptide
CPYKC

SEQ ID NO: 240 - protein, 5 aa, Artificial Sequence
  synthetic peptide
CPYRC

SEQ ID NO: 241 - protein, 5 aa, Artificial Sequence
  synthetic peptide
CPYHC

SEQ ID NO: 242 - protein, 5 aa, Artificial Sequence
  synthetic peptide
CPYGC

SEQ ID NO: 243 - protein, 5 aa, Artificial Sequence
  synthetic peptide
CPYAC

SEQ ID NO: 244 - protein, 5 aa, Artificial Sequence
  synthetic peptide
CPYVC

SEQ ID NO: 245 - protein, 5 aa, Artificial Sequence
  synthetic peptide
CPYLC

SEQ ID NO: 246 - protein, 5 aa, Artificial Sequence
  synthetic peptide
CPYIC

SEQ ID NO: 247 - protein, 5 aa, Artificial Sequence
  synthetic peptide
CPYMC

SEQ ID NO: 248 - protein, 5 aa, Artificial Sequence
  synthetic peptide
CPYFC

SEQ ID NO: 249 - protein, 5 aa, Artificial Sequence
  synthetic peptide
CPYWC

SEQ ID NO: 250 - protein, 5 aa, Artificial Sequence
  synthetic peptide
CPYPC

SEQ ID NO: 251 - protein, 5 aa, Artificial Sequence
  synthetic peptide
CPYSC

SEQ ID NO: 252 - protein, 5 aa, Artificial Sequence
  synthetic peptide
CPYTC

SEQ ID NO: 253 - protein, 5 aa, Artificial Sequence
  synthetic peptide
CPYCC

SEQ ID NO: 254 - protein, 5 aa, Artificial Sequence
  synthetic peptide
CPYYC

SEQ ID NO: 255 - protein, 5 aa, Artificial Sequence
  synthetic peptide
CPYNC

SEQ ID NO: 256 - protein, 5 aa, Artificial Sequence
  synthetic peptide
CPYQC

SEQ ID NO: 257 - protein, 5 aa, Artificial Sequence
  synthetic peptide
CPYDC

SEQ ID NO: 258 - protein, 5 aa, Artificial Sequence
  synthetic peptide
CPYEC

SEQ ID NO: 259 - protein, 5 aa, Artificial Sequence
  synthetic peptide
CPYLC

SEQ ID NO: 260 - protein, 5 aa, Artificial Sequence
  synthetic peptide
  misc_feature (2)..(2): Xaa can be any naturally occurring amino acid
CXHGC

SEQ ID NO: 261 - protein, 5 aa, Artificial Sequence
  synthetic peptide
  misc_feature (3)..(3): Xaa can be any naturally occurring amino acid
CHXGC

SEQ ID NO: 262 - protein, 5 aa, Artificial Sequence
  synthetic peptide
  misc_feature (4)..(4): Xaa can be any naturally occurring amino acid
CHGXC

SEQ ID NO: 263 - protein, 5 aa, Artificial Sequence
  synthetic peptide
CKHGC

SEQ ID NO: 264 - protein, 5 aa, Artificial Sequence
  synthetic peptide
CRHGC

SEQ ID NO: 265 - protein, 5 aa, Artificial Sequence
  synthetic peptide
CHHGC

SEQ ID NO: 266 - protein, 5 aa, Artificial Sequence
  synthetic peptide
CGHGC

SEQ ID NO: 267 - protein, 5 aa, Artificial Sequence
  synthetic peptide
CAHGC

SEQ ID NO: 268 - protein, 5 aa, Artificial Sequence
  synthetic peptide
CVHGC

SEQ ID NO: 269 - protein, 5 aa, Artificial Sequence
  synthetic peptide
CLHGC

SEQ ID NO: 270 - protein, 5 aa, Artificial Sequence
  synthetic peptide
CIHGC

SEQ ID NO: 271 - protein, 5 aa, Artificial Sequence
  synthetic peptide
CMHGC

SEQ ID NO: 272 - protein, 5 aa, Artificial Sequence
  synthetic peptide
CFHGC

SEQ ID NO: 273 - protein, 5 aa, Artificial Sequence
  synthetic peptide
CWHGC

SEQ ID NO: 274 - protein, 5 aa, Artificial Sequence
  synthetic peptide
CPHGC

SEQ ID NO: 275 - protein, 5 aa, Artificial Sequence
  synthetic peptide
CSHGC

SEQ ID NO: 276 - protein, 5 aa, Artificial Sequence
  synthetic peptide
CTHGC

SEQ ID NO: 277 - protein, 5 aa, Artificial Sequence
  synthetic peptide
CCHGC

SEQ ID NO: 278 - protein, 5 aa, Artificial Sequence
  synthetic peptide
CYHGC

SEQ ID NO: 279 - protein, 5 aa, Artificial Sequence
  synthetic peptide
CNHGC

SEQ ID NO: 280 - protein, 5 aa, Artificial Sequence
  synthetic peptide
CQHGC

SEQ ID NO: 281 - protein, 5 aa, Artificial Sequence
  synthetic peptide
CDHGC

SEQ ID NO: 282 - protein, 5 aa, Artificial Sequence
  synthetic peptide
CEHGC

SEQ ID NO: 283 - protein, 5 aa, Artificial Sequence
  synthetic peptide
CGKHC

SEQ ID NO: 284 - protein, 5 aa, Artificial Sequence
  synthetic peptide
CGRHC

SEQ ID NO: 285 - protein, 5 aa, Artificial Sequence
  synthetic peptide
CGHHC

SEQ ID NO: 286 - protein, 5 aa, Artificial Sequence
  synthetic peptide
CGGHC

SEQ ID NO: 287 - protein, 5 aa, Artificial Sequence
  synthetic peptide
CGAHC

SEQ ID NO: 288 - protein, 5 aa, Artificial Sequence
  synthetic peptide
CGVHC

SEQ ID NO: 289 - protein, 5 aa, Artificial Sequence
  synthetic peptide
CGLHC

SEQ ID NO: 290 - protein, 5 aa, Artificial Sequence
  synthetic peptide
CGIHC

SEQ ID NO: 291 - protein, 5 aa, Artificial Sequence
  synthetic peptide
CGMHC

SEQ ID NO: 292 - protein, 5 aa, Artificial Sequence
  synthetic peptide
CGFHC

SEQ ID NO: 293 - protein, 5 aa, Artificial Sequence
  synthetic peptide
CGWHC

SEQ ID NO: 294 - protein, 5 aa, Artificial Sequence
  synthetic peptide
CGPHC

SEQ ID NO: 295 - protein, 5 aa, Artificial Sequence
  synthetic peptide
CGSHC

SEQ ID NO: 296 - protein, 5 aa, Artificial Sequence
  synthetic peptide
CGTHC

SEQ ID NO: 297 - protein, 5 aa, Artificial Sequence
  synthetic peptide
CGCHC

SEQ ID NO: 298 - protein, 5 aa, Artificial Sequence
  synthetic peptide
CGYHC

SEQ ID NO: 299 - protein, 5 aa, Artificial Sequence
  synthetic peptide
CGNHC

SEQ ID NO: 300 - protein, 5 aa, Artificial Sequence
  synthetic peptide
CGQHC

SEQ ID NO: 301 - protein, 5 aa, Artificial Sequence
  synthetic peptide
CGDHC

SEQ ID NO: 302 - protein, 5 aa, Artificial Sequence
  synthetic peptide
CGEHC

SEQ ID NO: 303 - protein, 5 aa, Artificial Sequence
  synthetic peptide
CHGKC

SEQ ID NO: 304 - protein, 5 aa, Artificial Sequence
  synthetic peptide
CHGRC

SEQ ID NO: 305 - protein, 5 aa, Artificial Sequence
  synthetic peptide
CHGHC

SEQ ID NO: 306 - protein, 5 aa, Artificial Sequence
  synthetic peptide
CHGGC

SEQ ID NO: 307 - protein, 5 aa, Artificial Sequence
  synthetic peptide
CHGAC

SEQ ID NO: 308 - protein, 5 aa, Artificial Sequence
  synthetic peptide
CHGVC

SEQ ID NO: 309 - protein, 5 aa, Artificial Sequence
  synthetic peptide
CHGLC

SEQ ID NO: 310 - protein, 5 aa, Artificial Sequence
  synthetic peptide
CHGIC

SEQ ID NO: 311 - protein, 5 aa, Artificial Sequence
  synthetic peptide
CHGMC

SEQ ID NO: 312 - protein, 5 aa, Artificial Sequence
  synthetic peptide
CHGFC

SEQ ID NO: 313 - protein, 5 aa, Artificial Sequence
  synthetic peptide
CHGWC

SEQ ID NO: 314 - protein, 5 aa, Artificial Sequence
  synthetic peptide
CHGPC

SEQ ID NO: 315 - protein, 5 aa, Artificial Sequence
  synthetic peptide
CHGSC

SEQ ID NO: 316 - protein, 5 aa, Artificial Sequence
  synthetic peptide
CHGTC

SEQ ID NO: 317 - protein, 5 aa, Artificial Sequence
  synthetic peptide
CHGCC

SEQ ID NO: 318 - protein, 5 aa, Artificial Sequence
  synthetic peptide
CHGYC

SEQ ID NO: 319 - protein, 5 aa, Artificial Sequence
  synthetic peptide
CHGNC

SEQ ID NO: 320 - protein, 5 aa, Artificial Sequence
  synthetic peptide
CHGQC

SEQ ID NO: 321 - protein, 5 aa, Artificial Sequence
  synthetic peptide
CHGDC

SEQ ID NO: 322 - protein, 5 aa, Artificial Sequence
  synthetic peptide
CHGEC

SEQ ID NO: 323 - protein, 5 aa, Artificial Sequence
  synthetic peptide
  misc_feature (2)..(2): Xaa can be any naturally occurring amino acid
CXGPC

SEQ ID NO: 324 - protein, 5 aa, Artificial Sequence
  synthetic peptide
  misc_feature (3)..(3): Xaa can be any naturally occurring amino acid
CGXPC

SEQ ID NO: 325 - protein, 5 aa, Artificial Sequence
  synthetic peptide
  misc_feature (4)..(4): Xaa can be any naturally occurring amino acid
CGPXC

SEQ ID NO: 326 - protein, 5 aa, Artificial Sequence
  synthetic peptide
CKGPC

SEQ ID NO: 327 - protein, 5 aa, Artificial Sequence
  synthetic peptide
CRGPC

SEQ ID NO: 328 - protein, 5 aa, Artificial Sequence
  synthetic peptide
CHGPC

SEQ ID NO: 329 - protein, 5 aa, Artificial Sequence
  synthetic peptide
CGGPC

SEQ ID NO: 330 - protein, 5 aa, Artificial Sequence
  synthetic peptide
CAGPC

SEQ ID NO: 331 - protein, 5 aa, Artificial Sequence
  synthetic peptide
CVGPC

SEQ ID NO: 332 - protein, 5 aa, Artificial Sequence
  synthetic peptide
CLGPC

SEQ ID NO: 333 - protein, 5 aa, Artificial Sequence
  synthetic peptide
CIGPC

SEQ ID NO: 334 - protein, 5 aa, Artificial Sequence
  synthetic peptide
CMGPC

SEQ ID NO: 335 - protein, 5 aa, Artificial Sequence
  synthetic peptide
CFGPC

SEQ ID NO: 336 - protein, 5 aa, Artificial Sequence
  synthetic peptide
CWGPC

SEQ ID NO: 337 - protein, 5 aa, Artificial Sequence
  synthetic peptide
CPGPC

SEQ ID NO: 338 - protein, 5 aa, Artificial Sequence
  synthetic peptide
CSGPC

SEQ ID NO: 339 - protein, 5 aa, Artificial Sequence
  synthetic peptide
CTGPC

SEQ ID NO: 340 - protein, 5 aa, Artificial Sequence
  synthetic peptide
CCGPC

SEQ ID NO: 341 - protein, 5 aa, Artificial Sequence
  synthetic peptide
CYGPC

SEQ ID NO: 342 - protein, 5 aa, Artificial Sequence
  synthetic peptide
CNGPC

SEQ ID NO: 343 - protein, 5 aa, Artificial Sequence
  synthetic peptide
CQGPC

SEQ ID NO: 344 - protein, 5 aa, Artificial Sequence
  synthetic peptide
CDGPC

SEQ ID NO: 345 - protein, 5 aa, Artificial Sequence
  synthetic peptide
CEGPC

SEQ ID NO: 346 - protein, 5 aa, Artificial Sequence
  synthetic peptide
CGKPC

SEQ ID NO: 347 - protein, 5 aa, Artificial Sequence
  synthetic peptide
CGRPC

SEQ ID NO: 348 - protein, 5 aa, Artificial Sequence
  synthetic peptide
CGHPC

SEQ ID NO: 349 - protein, 5 aa, Artificial Sequence
  synthetic peptide
CGGPC

SEQ ID NO: 350 - protein, 5 aa, Artificial Sequence
  synthetic peptide
CGAPC

SEQ ID NO: 351 - protein, 5 aa, Artificial Sequence
  synthetic peptide
CGVPC

SEQ ID NO: 352 - protein, 5 aa, Artificial Sequence
  synthetic peptide
CGLPC

SEQ ID NO: 353 - protein, 5 aa, Artificial Sequence
  synthetic peptide
CGIPC

SEQ ID NO: 354 - protein, 5 aa, Artificial Sequence
  synthetic peptide
CGMPC

SEQ ID NO: 355 - protein, 5 aa, Artificial Sequence
  synthetic peptide
CGFPC

SEQ ID NO: 356 - protein, 5 aa, Artificial Sequence
  synthetic peptide
CGWPC

SEQ ID NO: 357 - protein, 5 aa, Artificial Sequence
  synthetic peptide
CGPPC

SEQ ID NO: 358 - protein, 5 aa, Artificial Sequence
  synthetic peptide
CGSPC

SEQ ID NO: 359 - protein, 5 aa, Artificial Sequence
  synthetic peptide
CGTPC

SEQ ID NO: 360 - protein, 5 aa, Artificial Sequence
  synthetic peptide
CGCPC

SEQ ID NO: 361 - protein, 5 aa, Artificial Sequence
  synthetic peptide
CGYPC

SEQ ID NO: 362 - protein, 5 aa, Artificial Sequence
  synthetic peptide
CGNPC

SEQ ID NO: 363 - protein, 5 aa, Artificial Sequence
  synthetic peptide
CGQPC

SEQ ID NO: 364 - protein, 5 aa, Artificial Sequence
  synthetic peptide
CGDPC

SEQ ID NO: 365 - protein, 5 aa, Artificial Sequence
  synthetic peptide
CGEPC

SEQ ID NO: 366 - protein, 5 aa, Artificial Sequence
  synthetic peptide
CGPKC

SEQ ID NO: 367 - protein, 5 aa, Artificial Sequence
  synthetic peptide
CGPRC

SEQ ID NO: 368 - protein, 5 aa, Artificial Sequence
  synthetic peptide
CGPHC

SEQ ID NO: 369 - protein, 5 aa, Artificial Sequence
  synthetic peptide
CGPGC

SEQ ID NO: 370 - protein, 5 aa, Artificial Sequence
  synthetic peptide
CGPAC

SEQ ID NO: 371 - protein, 5 aa, Artificial Sequence
  synthetic peptide
CGPVC

SEQ ID NO: 372 - protein, 5 aa, Artificial Sequence
  synthetic peptide
CGPLC

SEQ ID NO: 373 - protein, 5 aa, Artificial Sequence
  synthetic peptide
CGPIC

SEQ ID NO: 374 - protein, 5 aa, Artificial Sequence
  synthetic peptide
CGPMC

SEQ ID NO: 375 - protein, 5 aa, Artificial Sequence
  synthetic peptide
CGPFC

SEQ ID NO: 376 - protein, 5 aa, Artificial Sequence
  synthetic peptide
CGPWC

SEQ ID NO: 377 - protein, 5 aa, Artificial Sequence
  synthetic peptide
CGPPC

SEQ ID NO: 378 - protein, 5 aa, Artificial Sequence
  synthetic peptide
CGPSC

SEQ ID NO: 379 - protein, 5 aa, Artificial Sequence
  synthetic peptide
CGPTC

SEQ ID NO: 380 - protein, 5 aa, Artificial Sequence
  synthetic peptide
CGPCC

SEQ ID NO: 381 - protein, 5 aa, Artificial Sequence
  synthetic peptide
CGPYC

SEQ ID NO: 382 - protein, 5 aa, Artificial Sequence
  synthetic peptide
CGPNC

SEQ ID NO: 383 - protein, 5 aa, Artificial Sequence
  synthetic peptide
CGPQC

SEQ ID NO: 384 - protein, 5 aa, Artificial Sequence
  synthetic peptide
CGPDC

SEQ ID NO: 385 - protein, 5 aa, Artificial Sequence
  synthetic peptide
CGPEC

SEQ ID NO: 386 - protein, 5 aa, Artificial Sequence
  synthetic peptide
  misc_feature (2)..(2): Xaa can be any naturally occurring amino acid
CXGHC

SEQ ID NO: 387 - protein, 5 aa, Artificial Sequence
  synthetic peptide
  misc_feature (3)..(3): Xaa can be any naturally occurring amino acid
CGXHC

SEQ ID NO: 388 - protein, 5 aa, Artificial Sequence
  synthetic peptide
  misc_feature (4)..(4): Xaa can be any naturally occurring amino acid
CGHXC

SEQ ID NO: 389 - protein, 5 aa, Artificial Sequence
  synthetic peptide
CKGHC

SEQ ID NO: 390 - protein, 5 aa, Artificial Sequence
  synthetic peptide
CRGHC

SEQ ID NO: 391 - protein, 5 aa, Artificial Sequence
  synthetic peptide
CHGHC

SEQ ID NO: 392 - protein, 5 aa, Artificial Sequence
  synthetic peptide
CGGHC

SEQ ID NO: 393 - protein, 5 aa, Artificial Sequence
  synthetic peptide
CAGHC

SEQ ID NO: 394 - protein, 5 aa, Artificial Sequence
  synthetic peptide
CVGHC

SEQ ID NO: 395 - protein, 5 aa, Artificial Sequence
  synthetic peptide
CLGHC

SEQ ID NO: 396 - protein, 5 aa, Artificial Sequence
  synthetic peptide
CIGHC

SEQ ID NO: 397 - protein, 5 aa, Artificial Sequence
  synthetic peptide
CMGHC

SEQ ID NO: 398 - protein, 5 aa, Artificial Sequence
  synthetic peptide
CFGHC

SEQ ID NO: 399 - protein, 5 aa, Artificial Sequence
  synthetic peptide
CWGHC

SEQ ID NO: 400 - protein, 5 aa, Artificial Sequence
  synthetic peptide
CPGHC

SEQ ID NO: 401 - protein, 5 aa, Artificial Sequence
  synthetic peptide
CSGHC

SEQ ID NO: 402 - protein, 5 aa, Artificial Sequence
  synthetic peptide
CTGHC

SEQ ID NO: 403 - protein, 5 aa, Artificial Sequence
  synthetic peptide
CCGHC

SEQ ID NO: 404 - protein, 5 aa, Artificial Sequence
  synthetic peptide
CYGHC

SEQ ID NO: 405 - protein, 5 aa, Artificial Sequence
  synthetic peptide
CNGHC

SEQ ID NO: 406 - protein, 5 aa, Artificial Sequence
  synthetic peptides
CQGHC

SEQ ID NO: 407 - protein, 5 aa, Artificial Sequence
  synthetic peptide
CDGHC

SEQ ID NO: 408 - protein, 5 aa, Artificial Sequence
  synthetic peptide
CEGHC

SEQ ID NO: 409 - protein, 5 aa, Artificial Sequence
  synthetic peptide
  misc_feature (3)..(3): Xaa can be any naturally occurring amino acid
CGXFC

SEQ ID NO: 410 - protein, 5 aa, Artificial Sequence
  synthetic peptide
CGKFC

SEQ ID NO: 411 - protein, 5 aa, Artificial Sequence
  synthetic peptide
CGRFC

SEQ ID NO: 412 - protein, 5 aa, Artificial Sequence
  synthetic peptide
CGHFC

SEQ ID NO: 413 - protein, 5 aa, Artificial Sequence
  synthetic peptide
CGGFC

SEQ ID NO: 414 - protein, 5 aa, Artificial Sequence
  synthetic peptide
CGAFC

SEQ ID NO: 415 - protein, 5 aa, Artificial Sequence
  synthetic peptide
CGVFC

SEQ ID NO: 416 - protein, 5 aa, Artificial Sequence
  synthetic peptide
CGLFC

SEQ ID NO: 417 - protein, 5 aa, Artificial Sequence
  synthetic peptide
CGIFC

SEQ ID NO: 418 - protein, 5 aa, Artificial Sequence
  synthetic peptide
CGMFC

SEQ ID NO: 419 - protein, 5 aa, Artificial Sequence
  synthetic peptide
CGFFC

SEQ ID NO: 420 - protein, 5 aa, Artificial Sequence
  synthetic peptide
CGWFC

SEQ ID NO: 421 - protein, 5 aa, Artificial Sequence
  synthetic peptide
CGSFC

SEQ ID NO: 422 - protein, 5 aa, Artificial Sequence
  synthetic peptide
CGTFC

SEQ ID NO: 423 - protein, 5 aa, Artificial Sequence
  synthetic peptide
CGCFC

SEQ ID NO: 424 - protein, 5 aa, Artificial Sequence
  synthetic peptide
CGYFC

SEQ ID NO: 425 - protein, 5 aa, Artificial Sequence
  synthetic peptide
CGNFC

SEQ ID NO: 426 - protein, 5 aa, Artificial Sequence
  synthetic peptide
CGQFC

SEQ ID NO: 427 - protein, 5 aa, Artificial Sequence
  synthetic peptide
CGDFC

SEQ ID NO: 428 - protein, 5 aa, Artificial Sequence
  synthetic peptide
CGEFC

SEQ ID NO: 429 - protein, 5 aa, Artificial Sequence
  synthetic peptide
CGHKC

SEQ ID NO: 430 - protein, 5 aa, Artificial Sequence
  synthetic peptide
CGHRC

SEQ ID NO: 431 - protein, 5 aa, Artificial Sequence
  synthetic peptide
CGHHC

SEQ ID NO: 432 - protein, 5 aa, Artificial Sequence
  synthetic peptide
CGHGC

SEQ ID NO: 433 - protein, 5 aa, Artificial Sequence
  synthetic peptide
CGHAC

SEQ ID NO: 434 - protein, 5 aa, Artificial Sequence
  synthetic peptide
CGHVC

SEQ ID NO: 435 - protein, 5 aa, Artificial Sequence
  synthetic peptide
CGHLC

SEQ ID NO: 436 - protein, 5 aa, Artificial Sequence
  synthetic peptide
CGHIC

SEQ ID NO: 437 - protein, 5 aa, Artificial Sequence
  synthetic peptide
CGHMC

SEQ ID NO: 438 - protein, 5 aa, Artificial Sequence
  synthetic peptide
CGHWC

SEQ ID NO: 439 - protein, 5 aa, Artificial Sequence
  synthetic peptide
CGHPC

SEQ ID NO: 440 - protein, 5 aa, Artificial Sequence
  synthetic peptide
CGHSC

SEQ ID NO: 441 - protein, 5 aa, Artificial Sequence
  synthetic peptide
CGHTC

SEQ ID NO: 442 - protein, 5 aa, Artificial Sequence
  synthetic peptide
CGHCC

SEQ ID NO: 443 - protein, 5 aa, Artificial Sequence
  synthetic peptide
CGHYC

SEQ ID NO: 444 - protein, 5 aa, Artificial Sequence
  synthetic peptide
CGHNC

SEQ ID NO: 445 - protein, 5 aa, Artificial Sequence
  synthetic peptide
CGHQC

SEQ ID NO: 446 - protein, 5 aa, Artificial Sequence
  synthetic peptide
CGHDC

SEQ ID NO: 447 - protein, 5 aa, Artificial Sequence
  synthetic peptide
CGHEC

SEQ ID NO: 448 - protein, 5 aa, Artificial Sequence
  synthetic peptide
  misc_feature (2)..(2): Xaa can be any naturally occurring amino acid
CXGFC

SEQ ID NO: 449 - protein, 5 aa, Artificial Sequence
  synthetic peptide
  misc_feature (4)..(4): Xaa can be any naturally occurring amino acid
CGFXC

SEQ ID NO: 450 - protein, 5 aa, Artificial Sequence
  synthetic peptide
CKGFC

SEQ ID NO: 451 - protein, 5 aa, Artificial Sequence
  synthetic peptide
CRGFC

SEQ ID NO: 452 - protein, 5 aa, Artificial Sequence
  synthetic peptide
CHGFC

SEQ ID NO: 453 - protein, 5 aa, Artificial Sequence
  synthetic peptide
CAGFC

SEQ ID NO: 454 - protein, 5 aa, Artificial Sequence
  synthetic peptide
CVGFC

SEQ ID NO: 455 - protein, 5 aa, Artificial Sequence
  synthetic peptide
CLGFC

SEQ ID NO: 456 - protein, 5 aa, Artificial Sequence
  synthetic peptide
CIGFC

SEQ ID NO: 457 - protein, 5 aa, Artificial Sequence
  synthetic peptide
CMGFC

SEQ ID NO: 458 - protein, 5 aa, Artificial Sequence
  synthetic peptide
CFGFC

SEQ ID NO: 459 - protein, 5 aa, Artificial Sequence
  synthetic peptide
CWGFC

SEQ ID NO: 460 - protein, 5 aa, Artificial Sequence
  synthetic peptide
CPGFC

SEQ ID NO: 461 - protein, 5 aa, Artificial Sequence
  synthetic peptide
CSGFC

SEQ ID NO: 462 - protein, 5 aa, Artificial Sequence
  synthetic peptide
CTGFC

SEQ ID NO: 463 - protein, 5 aa, Artificial Sequence
  synthetic peptide
CCGFC

SEQ ID NO: 464 - protein, 5 aa, Artificial Sequence
  synthetic peptide
CYGFC

SEQ ID NO: 465 - protein, 5 aa, Artificial Sequence
  synthetic peptide
CNGFC

SEQ ID NO: 466 - protein, 5 aa, Artificial Sequence
  synthetic peptide
CQGFC

SEQ ID NO: 467 - protein, 5 aa, Artificial Sequence
  synthetic peptide
CDGFC

SEQ ID NO: 468 - protein, 5 aa, Artificial Sequence
  synthetic peptide
CEGFC

SEQ ID NO: 469 - protein, 5 aa, Artificial Sequence
  synthetic peptide
CGFKC

SEQ ID NO: 470 - protein, 5 aa, Artificial Sequence
  synthetic peptide
CGFRC

SEQ ID NO: 471 - protein, 5 aa, Artificial Sequence
  synthetic peptide
CGFHC

SEQ ID NO: 472 - protein, 5 aa, Artificial Sequence
  synthetic peptide
CGFGC

SEQ ID NO: 473 - protein, 5 aa, Artificial Sequence
  synthetic peptide
CGFAC

SEQ ID NO: 474 - protein, 5 aa, Artificial Sequence
  synthetic peptide
CGFVC

SEQ ID NO: 475 - protein, 5 aa, Artificial Sequence
  synthetic peptide
CGFLC

SEQ ID NO: 476 - protein, 5 aa, Artificial Sequence
  synthetic peptide
CGFIC

SEQ ID NO: 477 - protein, 5 aa, Artificial Sequence
  synthetic peptide
CGFMC

SEQ ID NO: 478 - protein, 5 aa, Artificial Sequence
  synthetic peptide
CGFWC

SEQ ID NO: 479 - protein, 5 aa, Artificial Sequence
  synthetic peptide
CGFPC

SEQ ID NO: 480 - protein, 5 aa, Artificial Sequence
  synthetic peptide
CGFSC

SEQ ID NO: 481 - protein, 5 aa, Artificial Sequence
  synthetic peptide
CGFTC

SEQ ID NO: 482 - protein, 5 aa, Artificial Sequence
  synthetic peptide
CGFCC

SEQ ID NO: 483 - protein, 5 aa, Artificial Sequence
  synthetic peptide
CGFYC

SEQ ID NO: 484 - protein, 5 aa, Artificial Sequence
  synthetic peptide
CGFNC

SEQ ID NO: 485 - protein, 5 aa, Artificial Sequence
  synthetic peptide
CGFQC

SEQ ID NO: 486 - protein, 5 aa, Artificial Sequence
  synthetic peptide
CGFDC

SEQ ID NO: 487 - protein, 5 aa, Artificial Sequence
  synthetic peptide
CGFEC

SEQ ID NO: 488 - protein, 5 aa, Artificial Sequence
  synthetic peptide
  misc_feature (2)..(2): Xaa can be any naturally occurring amino acid
CXRLC

SEQ ID NO: 489 - protein, 5 aa, Artificial Sequence
  synthetic peptide
  misc_feature (3)..(3): Xaa can be any naturally occurring amino acid
CRXLC

SEQ ID NO: 490 - protein, 5 aa, Artificial Sequence
  synthetic peptide
  misc_feature (4)..(4): Xaa can be any naturally occurring amino acid
CRLXC

SEQ ID NO: 491 - protein, 5 aa, Artificial Sequence
  synthetic peptide
CKRLC

SEQ ID NO: 492 - protein, 5 aa, Artificial Sequence
  synthetic peptide
CRRLC

SEQ ID NO: 493 - protein, 5 aa, Artificial Sequence
  synthetic peptide
CHRLC

SEQ ID NO: 494 - protein, 5 aa, Artificial Sequence
  synthetic peptide
CGRLC

SEQ ID NO: 495 - protein, 5 aa, Artificial Sequence
  synthetic peptide
CARLC

SEQ ID NO: 496 - protein, 5 aa, Artificial Sequence
  synthetic peptide
CVRLC

SEQ ID NO: 497 - protein, 5 aa, Artificial Sequence
  synthetic peptide
CLRLC

SEQ ID NO: 498 - protein, 5 aa, Artificial Sequence
  synthetic peptide
CIRLC

SEQ ID NO: 499 - protein, 5 aa, Artificial Sequence
  synthetic peptide
CMRLC

SEQ ID NO: 500 - protein, 5 aa, Artificial Sequence
  synthetic peptide
CFRLC

SEQ ID NO: 501 - protein, 5 aa, Artificial Sequence
  synthetic peptide
CWRLC

SEQ ID NO: 502 - protein, 5 aa, Artificial Sequence
  synthetic peptide
CPRLC

SEQ ID NO: 503 - protein, 5 aa, Artificial Sequence
  synthetic peptide
CSRLC

SEQ ID NO: 504 - protein, 5 aa, Artificial Sequence
  synthetic peptide
CTRLC

SEQ ID NO: 505 - protein, 5 aa, Artificial Sequence
  synthetic peptide
CCRLC

SEQ ID NO: 506 - protein, 5 aa, Artificial Sequence
  synthetic peptide
CYRLC

SEQ ID NO: 507 - protein, 5 aa, Artificial Sequence
  synthetic peptide
CNRLC

SEQ ID NO: 508 - protein, 5 aa, Artificial Sequence
  synthetic peptide
CQRLC

SEQ ID NO: 509 - protein, 5 aa, Artificial Sequence
  synthetic peptide
CDRLC

SEQ ID NO: 510 - protein, 5 aa, Artificial Sequence
  synthetic peptide
CERLC

SEQ ID NO: 511 - protein, 5 aa, Artificial Sequence
  synthetic peptide
CRKLC

SEQ ID NO: 512 - protein, 5 aa, Artificial Sequence
  synthetic peptide
CRRLC

SEQ ID NO: 513 - protein, 5 aa, Artificial Sequence
  synthetic peptide
CRHLC

SEQ ID NO: 514 - protein, 5 aa, Artificial Sequence
  synthetic peptide
CRGLC

SEQ ID NO: 515 - protein, 5 aa, Artificial Sequence
  synthetic peptide
CRALC

SEQ ID NO: 516 - protein, 5 aa, Artificial Sequence
  synthetic peptide
CRVLC

SEQ ID NO: 517 - protein, 5 aa, Artificial Sequence
  synthetic peptide
CRLLC

SEQ ID NO: 518 - protein, 5 aa, Artificial Sequence
  synthetic peptide
CRILC

SEQ ID NO: 519 - protein, 5 aa, Artificial Sequence
  synthetic peptide
CRMLC

SEQ ID NO: 520 - protein, 5 aa, Artificial Sequence
  synthetic peptide
CRFLC

SEQ ID NO: 521 - protein, 5 aa, Artificial Sequence
  synthetic peptide
CRWLC

SEQ ID NO: 522 - protein, 5 aa, Artificial Sequence
  synthetic peptide
CRPLC

SEQ ID NO: 523 - protein, 5 aa, Artificial Sequence
  synthetic peptide
CRSLC

SEQ ID NO: 524 - protein, 5 aa, Artificial Sequence
  synthetic peptide
CRTLC

SEQ ID NO: 525 - protein, 5 aa, Artificial Sequence
  synthetic peptide
CRCLC

SEQ ID NO: 526 - protein, 5 aa, Artificial Sequence
  synthetic peptide
CRYLC

SEQ ID NO: 527 - protein, 5 aa, Artificial Sequence
  synthetic peptide
CRNLC

SEQ ID NO: 528 - protein, 5 aa, Artificial Sequence
  synthetic peptide
CRQLC

SEQ ID NO: 529 - protein, 5 aa, Artificial Sequence
  synthetic peptide
CRDLC

SEQ ID NO: 530 - protein, 5 aa, Artificial Sequence
  synthetic peptide
CRELC

SEQ ID NO: 531 - protein, 5 aa, Artificial Sequence
  synthetic peptide
CRLKC

SEQ ID NO: 532 - protein, 5 aa, Artificial Sequence
  synthetic peptide
CRLRC

SEQ ID NO: 533 - protein, 5 aa, Artificial Sequence
  synthetic peptide
CRLHC

SEQ ID NO: 534 - protein, 5 aa, Artificial Sequence
  synthetic peptide
CRLGC

SEQ ID NO: 535 - protein, 5 aa, Artificial Sequence
  synthetic peptide
CRLAC

SEQ ID NO: 536 - protein, 5 aa, Artificial Sequence
  synthetic peptide
CRLVC

SEQ ID NO: 537 - protein, 5 aa, Artificial Sequence
  synthetic peptide
CRLIC

SEQ ID NO: 538 - protein, 5 aa, Artificial Sequence
  synthetic peptide
CRLMC

SEQ ID NO: 539 - protein, 5 aa, Artificial Sequence
  synthetic peptide
CRLFC

SEQ ID NO: 540 - protein, 5 aa, Artificial Sequence
  synthetic peptide
CRLWC

SEQ ID NO: 541 - protein, 5 aa, Artificial Sequence
  synthetic peptide
CRLPC

SEQ ID NO: 542 - protein, 5 aa, Artificial Sequence
  synthetic peptide
CRLSC

SEQ ID NO: 543 - protein, 5 aa, Artificial Sequence
  synthetic peptide
CRLTC

SEQ ID NO: 544 - protein, 5 aa, Artificial Sequence
  synthetic peptide
CRLCC

SEQ ID NO: 545 - protein, 5 aa, Artificial Sequence
  synthetic peptide
CRLYC

SEQ ID NO: 546 - protein, 5 aa, Artificial Sequence
  synthetic peptide
CRLNC

SEQ ID NO: 547 - protein, 5 aa, Artificial Sequence
  synthetic peptide
CRLQC

SEQ ID NO: 548 - protein, 5 aa, Artificial Sequence
  synthetic peptide
CRLDC

SEQ ID NO: 549 - protein, 5 aa, Artificial Sequence
  synthetic peptide
CRLEC

SEQ ID NO: 550 - protein, 5 aa, Artificial Sequence
  synthetic peptide
  misc_feature (2)..(2): Xaa can be any naturally occurring amino acid
CXHPC

SEQ ID NO: 551 - protein, 5 aa, Artificial Sequence
  synthetic peptide
  misc_feature (3)..(3): Xaa can be any naturally occurring amino acid
CHXPC

SEQ ID NO: 552 - protein, 5 aa, Artificial Sequence
  synthetic peptide
  misc_feature (4)..(4): Xaa can be any naturally occurring amino acid
CHPXC

SEQ ID NO: 553 - protein, 5 aa, Artificial Sequence
  synthetic peptide
CKHPC

SEQ ID NO: 554 - protein, 5 aa, Artificial Sequence
  synthetic peptide
CRHPC

SEQ ID NO: 555 - protein, 5 aa, Artificial Sequence
  synthetic peptide
CHHPC

SEQ ID NO: 556 - protein, 5 aa, Artificial Sequence
  synthetic peptide
CGHPC

SEQ ID NO: 557 - protein, 5 aa, Artificial Sequence
  synthetic peptide
CAHPC

SEQ ID NO: 558 - protein, 5 aa, Artificial Sequence
  synthetic peptide
CVHPC

SEQ ID NO: 559 - protein, 5 aa, Artificial Sequence
  synthetic peptide
CLHPC

SEQ ID NO: 560 - protein, 5 aa, Artificial Sequence
  synthetic peptide
CIHPC

SEQ ID NO: 561 - protein, 5 aa, Artificial Sequence
  synthetic peptide
CMHPC

SEQ ID NO: 562 - protein, 5 aa, Artificial Sequence
  synthetic peptide
CFHPC

SEQ ID NO: 563 - protein, 5 aa, Artificial Sequence
  synthetic peptide
CWHPC

SEQ ID NO: 564 - protein, 5 aa, Artificial Sequence
  synthetic peptide
CPHPC

SEQ ID NO: 565 - protein, 5 aa, Artificial Sequence
  synthetic peptide
CSHPC

SEQ ID NO: 566 - protein, 5 aa, Artificial Sequence
  synthetic peptide
CTHPC

SEQ ID NO: 567 - protein, 5 aa, Artificial Sequence
  synthetic peptide
CCHPC

SEQ ID NO: 568 - protein, 5 aa, Artificial Sequence
  synthetic peptide
CYHPC

SEQ ID NO: 569 - protein, 5 aa, Artificial Sequence
  synthetic peptide
CNHPC

SEQ ID NO: 570 - protein, 5 aa, Artificial Sequence
  synthetic peptide
CQHPC

SEQ ID NO: 571 - protein, 5 aa, Artificial Sequence
  synthetic peptide
CDHPC

SEQ ID NO: 572 - protein, 5 aa, Artificial Sequence
  synthetic peptide
CEHPC

SEQ ID NO: 573 - protein, 5 aa, Artificial Sequence
  synthetic peptide
CKHPC

SEQ ID NO: 574 - protein, 5 aa, Artificial Sequence
  synthetic peptide
CHKPC

SEQ ID NO: 575 - protein, 5 aa, Artificial Sequence
  synthetic peptide
CHRPC

SEQ ID NO: 576 - protein, 5 aa, Artificial Sequence
  synthetic peptide
CHHPC

SEQ ID NO: 577 - protein, 5 aa, Artificial Sequence
  synthetic peptide
CHGPC

SEQ ID NO: 578 - protein, 5 aa, Artificial Sequence
  synthetic peptide
CHAPC

SEQ ID NO: 579 - protein, 5 aa, Artificial Sequence
  synthetic peptide
CHVPC

SEQ ID NO: 580 - protein, 5 aa, Artificial Sequence
  synthetic peptide
CHLPC

SEQ ID NO: 581 - protein, 5 aa, Artificial Sequence
  synthetic peptide
CHIPC

SEQ ID NO: 582 - protein, 5 aa, Artificial Sequence
  synthetic peptide
CHMPC

SEQ ID NO: 583 - protein, 5 aa, Artificial Sequence
  synthetic peptide
CHFPC

SEQ ID NO: 584 - protein, 5 aa, Artificial Sequence
  synthetic peptide
CHWPC

SEQ ID NO: 585 - protein, 5 aa, Artificial Sequence
  synthetic peptide
CHPPC

SEQ ID NO: 586 - protein, 5 aa, Artificial Sequence
  synthetic peptide
CHSPC

SEQ ID NO: 587 - protein, 5 aa, Artificial Sequence
  synthetic peptide
CHTPC

SEQ ID NO: 588 - protein, 5 aa, Artificial Sequence
  synthetic peptide
CHCPC

SEQ ID NO: 589 - protein, 5 aa, Artificial Sequence
  synthetic peptide
CHYPC

SEQ ID NO: 590 - protein, 5 aa, Artificial Sequence
  synthetic peptide
CHNPC

SEQ ID NO: 591 - protein, 5 aa, Artificial Sequence
  synthetic peptide
CHQPC

SEQ ID NO: 592 - protein, 5 aa, Artificial Sequence
  synthetic peptide
CHDPC

SEQ ID NO: 593 - protein, 5 aa, Artificial Sequence
  synthetic peptide
CHEPC

SEQ ID NO: 594 - protein, 5 aa, Artificial Sequence
  synthetic peptide
CHPKC

SEQ ID NO: 595 - protein, 5 aa, Artificial Sequence
  synthetic peptide
CHPRC

SEQ ID NO: 596 - protein, 5 aa, Artificial Sequence
  synthetic peptide
CHPHC

SEQ ID NO: 597 - protein, 5 aa, Artificial Sequence
  synthetic peptide
CHPGC

SEQ ID NO: 598 - protein, 5 aa, Artificial Sequence
  synthetic peptide
CHPAC

SEQ ID NO: 599 - protein, 5 aa, Artificial Sequence
  synthetic peptide
CHPVC

SEQ ID NO: 600 - protein, 5 aa, Artificial Sequence
  synthetic peptide
CHPLC

SEQ ID NO: 601 - protein, 5 aa, Artificial Sequence
  synthetic peptide
CHPIC

SEQ ID NO: 602 - protein, 5 aa, Artificial Sequence
  synthetic peptide
CHPMC

SEQ ID NO: 603 - protein, 5 aa, Artificial Sequence
  synthetic peptide
CHPFC

SEQ ID NO: 604 - protein, 5 aa, Artificial Sequence
  synthetic peptide
CHPWC

SEQ ID NO: 605 - protein, 5 aa, Artificial Sequence
  synthetic peptide
CHPPC

SEQ ID NO: 606 - protein, 5 aa, Artificial Sequence
  synthetic peptide
CHPSC

SEQ ID NO: 607 - protein, 5 aa, Artificial Sequence
  synthetic peptide
CHPTC

SEQ ID NO: 608 - protein, 5 aa, Artificial Sequence
  synthetic peptide
CHPCC

SEQ ID NO: 609 - protein, 5 aa, Artificial Sequence
  synthetic peptide
CHPYC

SEQ ID NO: 610 - protein, 5 aa, Artificial Sequence
  synthetic peptide
CHPNC

SEQ ID NO: 611 - protein, 5 aa, Artificial Sequence
  synthetic peptide
CHPQC

SEQ ID NO: 612 - protein, 5 aa, Artificial Sequence
  synthetic peptide
CHPDC

SEQ ID NO: 613 - protein, 5 aa, Artificial Sequence
  synthetic peptide
CHPEC

SEQ ID NO: 614 - protein, 6 aa, Artificial Sequence
  synthetic peptide
  misc_feature (2)..(5): Xaa can be any naturally occurring amino acid
CXXXXC

SEQ ID NO: 615 - protein, 6 aa, Artificial Sequence
  synthetic peptide
CLAVLC

SEQ ID NO: 616 - protein, 6 aa, Artificial Sequence
  synthetic peptide
CTVQAC

SEQ ID NO: 617 - protein, 6 aa, Artificial Sequence
  synthetic peptide
CGAVHC

SEQ ID NO: 618 - protein, 6 aa, Artificial Sequence
  synthetic peptide
  misc_feature (2)..(2): Xaa can be any naturally occurring amino acid
CXAVLC

SEQ ID NO: 619 - protein, 6 aa, Artificial Sequence
  synthetic peptide
  misc_feature (3)..(3): Xaa can be any naturally occurring amino acid
CLXVLC

SEQ ID NO: 620 - protein, 6 aa, Artificial Sequence
  synthetic peptide
  misc_feature (4)..(4): Xaa can be any naturally occurring amino acid
CLAXLC

SEQ ID NO: 621 - protein, 6 aa, Artificial Sequence
  synthetic peptide
  misc_feature (5)..(5): Xaa can be any naturally occurring amino acid
CLAVXC

SEQ ID NO: 622 - protein, 6 aa, Artificial Sequence
  synthetic peptide
  misc_feature (2)..(2): Xaa can be any naturally occurring amino acid
CXVQAC

SEQ ID NO: 623 - protein, 6 aa, Artificial Sequence
  synthetic peptide
  misc_feature (3)..(3): Xaa can be any naturally occurring amino acid
CTXQAC

SEQ ID NO: 624 - protein, 6 aa, Artificial Sequence
  synthetic peptide
  misc_feature (4)..(4): Xaa can be any naturally occurring amino acid
CTVXAC

SEQ ID NO: 625 - protein, 6 aa, Artificial Sequence
  synthetic peptide
  misc_feature (5)..(5): Xaa can be any naturally occurring amino acid
CTVQXC

SEQ ID NO: 626 - protein, 6 aa, Artificial Sequence
  synthetic peptide
  misc_feature (2)..(2): Xaa can be any naturally occurring amino acid
CXAVHC

SEQ ID NO: 627 - protein, 6 aa, Artificial Sequence
  synthetic peptide
  misc_feature (3)..(3): Xaa can be any naturally occurring amino acid
CGXVHC

SEQ ID NO: 628 - protein, 6 aa, Artificial Sequence
  synthetic peptide
  misc_feature (4)..(4): Xaa can be any naturally occurring amino acid
CGAXHC

SEQ ID NO: 629 - protein, 6 aa, Artificial Sequence
  synthetic peptide
  misc_feature (5)..(5): Xaa can be any naturally occurring amino acid
CGAVXC

SEQ ID NO: 630 - protein, 7 aa, Artificial Sequence
  synthetic peptide
  misc_feature (2)..(6): Xaa can be any naturally occurring amino acid
CXXXXXC

SEQ ID NO: 631 - protein, 7 aa, Artificial Sequence
  synthetic peptide
CPAFPLC

SEQ ID NO: 632 - protein, 7 aa, Artificial Sequence
  synthetic peptide
CDQGGEC

SEQ ID NO: 633 - protein, 7 aa, Artificial Sequence
  synthetic peptide
  misc_feature (2)..(2): Xaa can be any naturally occurring amino acid
CXAFPLC

SEQ ID NO: 634 - protein, 7 aa, Artificial Sequence
  synthetic peptide
  misc_feature (3)..(3): Xaa can be any naturally occurring amino acid
CPXFPLC

SEQ ID NO: 635 - protein, 7 aa, Artificial Sequence
  synthetic peptide
  misc_feature (4)..(4): Xaa can be any naturally occurring amino acid
CPAXPLC

SEQ ID NO: 636 - protein, 7 aa, Artificial Sequence
  synthetic peptide
  misc_feature (5)..(5): Xaa can be any naturally occurring amino acid
CPAFXLC

SEQ ID NO: 637 - protein, 7 aa, Artificial Sequence
  synthetic peptide
  misc_feature (6)..(6): Xaa can be any naturally occurring amino acid
CPAFPXC

SEQ ID NO: 638 - protein, 7 aa, Artificial Sequence
  synthetic peptide
  misc_feature (2)..(2): Xaa can be any naturally occurring amino acid
CXQGGEC

SEQ ID NO: 639 - protein, 7 aa, Artificial Sequence
  synthetic peptide
  misc_feature (3)..(3): Xaa can be any naturally occurring amino acid
CDXGGEC

SEQ ID NO: 640 - protein, 7 aa, Artificial Sequence
  synthetic peptide
  misc_feature (4)..(4): Xaa can be any naturally occurring amino acid
CDQXGEC

SEQ ID NO: 641 - protein, 7 aa, Artificial Sequence
  synthetic peptide
  misc_feature (5)..(5): Xaa can be any naturally occurring amino acid
CDQGXEC

SEQ ID NO: 642 - protein, 7 aa, Artificial Sequence
  synthetic peptide
  misc_feature (6)..(6): Xaa can be any naturally occurring amino acid
CDQGGXC

SEQ ID NO: 643 - protein, 8 aa, Artificial Sequence
  synthetic peptide
  misc_feature (2)..(7): Xaa can be any naturally occurring amino acid
CXXXXXXC

SEQ ID NO: 644 - protein, 8 aa, Artificial Sequence
  synthetic peptide
CDIADKYC

SEQ ID NO: 645 - protein, 8 aa, Artificial Sequence
  synthetic peptide
  misc_feature (2)..(2): Xaa can be any naturally occurring amino acid
CXIADKYC

SEQ ID NO: 646 - protein, 8 aa, Artificial Sequence
  synthetic peptide
  misc_feature (3)..(3): Xaa can be any naturally occurring amino acid
CDXADKYC

SEQ ID NO: 647 - protein, 8 aa, Artificial Sequence
  synthetic peptide
  misc_feature (4)..(4): Xaa can be any naturally occurring amino acid
CDIXDKYC

SEQ ID NO: 648 - protein, 8 aa, Artificial Sequence
  synthetic peptide
  misc_feature (5)..(5): Xaa can be any naturally occurring amino acid
CDIAXKYC

SEQ ID NO: 649 - protein, 8 aa, Artificial Sequence
  synthetic peptide
  misc_feature (6)..(6): Xaa can be any naturally occurring amino acid
CDIADXYC

SEQ ID NO: 650 - protein, 8 aa, Artificial Sequence
  synthetic peptide
  misc_feature (7)..(7): Xaa can be any naturally occurring amino acid
CDIADKXC

SEQ ID NO: 651 - protein, 5 aa, Artificial Sequence
  synthetic peptide
  misc_feature (2)..(2): Xaa can be any naturally occurring amino acid
  PEPTIDE (3)..(3): K, H, or R
  misc_feature (4)..(4): Xaa can be any naturally occurring amino acid
CXXXC

SEQ ID NO: 652 - protein, 5 aa, Artificial Sequence
  synthetic peptide
  misc_feature (2)..(3): Xaa can be any naturally occurring amino acid
  PEPTIDE (4)..(4): K, H, or R
CXXXC

SEQ ID NO: 653 - protein, 4 aa, Artificial Sequence
  synthetic peptide
  PEPTIDE (1)..(1): K, H, or R
  misc_feature (3)..(3): Xaa can be any naturally occurring amino acid
XCXC

SEQ ID NO: 654 - protein, 5 aa, Artificial Sequence
  synthetic peptide
  PEPTIDE (1)..(1): K, H, or R
  misc_feature (2)..(4): Xaa can be any naturally occurring amino acid
XXXXC

SEQ ID NO: 655 - protein, 4 aa, Artificial Sequence
  synthetic peptide
  misc_feature (2)..(2): Xaa can be any naturally occurring amino acid
  PEPTIDE (4)..(4): K, H, or R
CXCX

SEQ ID NO: 656 - protein, 6 aa, Artificial Sequence
  synthetic peptide
  misc_feature (2)..(4): Xaa can be any naturally occurring amino acid
  PEPTIDE (6)..(6): K, H, or R
CXXXCX

SEQ ID NO: 657 - protein, 4 aa, Artificial Sequence
  synthetic peptide
  PEPTIDE (1)..(1): K, H, or R
  PEPTIDE (4)..(4): K, H, or R
XCCX

SEQ ID NO: 658 - protein, 5 aa, Artificial Sequence
  synthetic peptide
  PEPTIDE (1)..(1): K, H, or R
  misc_feature (3)..(3): Xaa can be any naturally occurring amino acid
  PEPTIDE (5)..(5): K, H, or R
XCXCX

SEQ ID NO: 659 - protein, 7 aa, Artificial Sequence
  synthetic peptide
  PEPTIDE (1)..(1): K, H, or R
  misc_feature (3)..(5): Xaa can be any naturally occurring amino acid
  PEPTIDE (7)..(7): K, H, or R
XCXXXCX

SEQ ID NO: 660 - protein, 4 aa, Artificial Sequence
  synthetic peptide
  PEPTIDE (1)..(1): K, H, or R
  PEPTIDE (3)..(3): K, H, or R
XCXC

SEQ ID NO: 661 - protein, 4 aa, Artificial Sequence
  synthetic peptide
  PEPTIDE (2)..(2): K, H, or R
  PEPTIDE (4)..(4): K, H, or R
CXCX

SEQ ID NO: 662 - protein, 6 aa, Artificial Sequence
  synthetic peptide
  PEPTIDE (1)..(1): K, H, or R
  misc_feature (3)..(4): Xaa can be any naturally occurring amino acid
  PEPTIDE (5)..(5): K, H, or R
XCXXXC

SEQ ID NO: 663 - protein, 6 aa, Artificial Sequence
  synthetic peptide
  PEPTIDE (1)..(1): K, H, or R
  misc_feature (3)..(3): Xaa can be any naturally occurring amino acid
  PEPTIDE (4)..(4): K, H, or R
  misc_feature (5)..(5): Xaa can be any naturally occurring amino acid
XCXXXC

SEQ ID NO: 664 - protein, 6 aa, Artificial Sequence
  synthetic peptide
  misc_feature (2)..(3): Xaa can be any naturally occurring amino acid
  PEPTIDE (4)..(4): K, H, or R
  PEPTIDE (6)..(6): K, H, or R
CXXXCX

SEQ ID NO: 665 - protein, 6 aa, Artificial Sequence
  synthetic peptide
  misc_feature (2)..(4): Xaa can be any naturally occurring amino acid
  misc_feature (6)..(6): Xaa can be any naturally occurring amino acid
CXXXCX

SEQ ID NO: 666 - protein, 6 aa, Artificial Sequence
  synthetic peptide
  PEPTIDE (2)..(2): K, H, or R
  misc_feature (3)..(4): Xaa can be any naturally occurring amino acid
  PEPTIDE (6)..(6): K, H, or R
CXXXCX

(19) United States
(12) Patent Application Publication
(10) Pub. No.: US 2023/0136112 A1
(43) Pub. Date: May 4, 2023

(54) METHODS FOR STRATIFYING DIABETES PATIENTS

(71) Applicant: IMCYSE SA, Angleur (Liége) (BE)

(72) Inventor: Vincent CARLIER, Enines (BE)

(21) Appl. No.: 17/780,141

(22) PCT Filed: Nov. 27, 2020
(86) PCT No.: PCT/EP2020/083642

§ 371 Date: May 26, 2022

(30) Foreign Application Priority Data
Nov. 27, 2019 (EP) 19211796.8

(51) Int. Cl. A61K 39/00; G01N 33/569; C07K 14/62; A61P 3/10; C12N 9/02

(52) U.S. Cl. CPC A61K 39/0008; G01N 33/56977; C07K 14/62; A61P 3/10; C12N 9/0004; A61K 2039/545; G01N 2800/52; G01N 2333/70539; C07K 2319/00

(57) ABSTRACT

The present invention relates to a method of predicting the response of of type 1 diabetes patients to the treatment with an immunogenic peptide comprising an MHCII T cell epitope of insulin and an oxidoreductase motif, said method comprising determining the MHC class II HLA haplotype of the patient.

This application is the U.S. national phase of International Application No. PCT/EP2020/083642 filed Nov. 27, 2020 which designated the U.S. and claims priority to EP 19211796.8 filed Nov. 27, 2019, the entire contents of each of which are hereby incorporated by reference.

### BACKGROUND OF THE INVENTION

Several strategies have been described to prevent the generation of an unwanted immune response against an antigen. WO2008/017517 describes a new strategy using peptides comprising an MHC class II T cell epitope of a given antigenic protein and an oxidoreductase motif. These peptides convert CD4+ T cells into a cell type with cytolytic properties called cytolytic CD4+ T cells. These cells are capable to kill via triggering apoptosis those antigen presenting cells (APC), which present the antigen from which the peptide is derived. WO2008/017517 demonstrates this concept for allergies and auto-immune diseases such as type 1 diabetes. Herein insulin can act as an auto-antigen.

WO2009101207 and Carlier et al. (2012) Plos one 7,10 e45366 further describe the antigen specific cytolytic cells in more detail.

WO2016059236 discloses further modified peptides wherein an additional histidine is present in the proximity of the oxidoreductase motif.

WO2018162498 further discloses a peptide comprising an oxidoreductase motif with an additional histidine and a MHCII T cell epitope from insulin and its use in the treatment of type 1 diabetes (T1D).

However, even when taking into account the above, there remains a need for a method of stratifying and selecting those T1D patient subpopulations that will benefit most of the treatment and if needed to tailor said treatment for other patient subpopulations that are less responsive. So far, no information was available regarding the effect of the use of the immunogenic peptides comprising an insulin T-cell epitope coupled to an oxidoreductase motif in T1D patients and the patient response thereto.

The present invention has identified a method to stratify T1D patients for likelihood of good response.

### SUMMARY OF THE INVENTION

The present invention provides a stratification method and tool for predicting responsiveness of type 1 diabetes patients to treatment with immunogenic peptides comprising an insulin antigen and an oxidoreductase motif and methods for the treatment of such type 1 diabetes patients with said immunogenic peptide.

Yet, the inventors have found that in patients, said level of responsiveness can be depending on the MHC class II haplotype.

The invention hence provides the following aspects:
1. An in vitro method for predicting the response of type 1 diabetes patients to the treatment with an immunogenic peptide with a length of between 12 and 50 amino acids, comprising an oxidoreductase motif and, separated from this motif by 0 to 7 amino acids, a (pro-)insulin MHC class II T cell epitope sequence, comprising determining the MHC class II HLA haplotype of the patient, wherein patients being HLA-DR4 positive (HLA-DR4+), are predicted to be responsive to said treatment.
   The term “HLA DR4 positive” encompasses both patients being heterozygous or homozygous HLA-DR4 positive.
   In one embodiment, said patients are also HLA-DR3 negative (HLA-DR3−).
   The term “HLA-DR3 negative” refers to patients being homozygous HLA-DR3 negative.
   More particularly, said stratification method identifies the patients that will benefit particularly well from treatment with the immunogenic peptides according to the invention.
   More specifically, said stratification method identifies patients that have an HLA-DR4 positive haplotype and optionally HLA-DR3 negative haplotype, as being more responsive over patients being HLA-DR4 negative.
   This responsiveness can e.g. be determined:
      by counting the total daily insulin doses per kg, wherein a reduction in said total number versus non-treated or non-responsive patients indicates a positive response to the treatment; or
      by using an MMTT test measuring C-peptide secretion, wherein responsive patients present a tendency to improvement (median decrease in C-peptide is slower than the reference model, delta ratio is >0) as compared to non-treated or non-responsive patients. In some embodiments, said haplotype determination in said patients is performed using polymerase chain reaction (PCR)-based analysis, sequence analysis, electrophoretic analysis or through antibody testing.
2. A method of reducing the immune response to an auto-antigen selected from (pro)insulin or C-peptide in a patient, comprising administering an immunogenic peptide with a length of between 12 and 50 amino acids, comprising an oxidoreductase motif and, separated from this motif by 0 to 7 amino acids, a (pro-)insulin MHC class II T cell epitope sequence, wherein said patient has been selected based on the presence of a DR4 positive (HLA-DR4+) and optionally HLA-DR3 negative (HLA-DR3−) MHC class II HLA haplotype.
3. An immunogenic peptide with a length of between 12 and 50 amino acids, comprising an oxidoreductase motif and, separated from this motif by 0 to 7 amino acids, a (pro-)insulin MHC class II T cell epitope sequence for use in the treatment or prevention of type 1 diabetes in patients selected based on the presence of a DR4 positive (HLA-DR4+) and optionally HLA-DR3 negative (HLA-DR3−) MHC class II HLA haplotype.
4. A method of treatment or prevention of type 1 diabetes comprising administering an effective dosage of an immunogenic peptide with a length of between 12 and 50 amino acids, comprising an oxidoreductase motif and, separated from this motif by 0 to 7 amino acids, a (pro-)insulin MHC class II T cell epitope sequence, to a patient selected based on the presence of a DR4 positive (HLA-DR4+) and optionally HLA-DR3 negative (HLA-DR3−) MHC class II HLA haplotype.
5. In certain embodiments of aspects 2 to 4, the MHC class II haplotype of said patients has been determined prior to treatment or is being determined during treatment.
   In some embodiments, said haplotype determination is performed using polymerase chain reaction (PCR)-based analysis, sequence analysis, electrophoretic analysis or through antibody testing.
6. In certain embodiments of any one of aspects 1 to 5, said oxidoreductase motif can comprise the general formula:

Zm[CST]XnC or ZmCXn[CST],
wherein n is an integer from 0 to 6, preferably 0 to 3, more preferably 0, 1, 2, or 3,
wherein m stands for an integer from 0 to 2,
in which C stands for cysteine, S for serine, T for threonine, X for any amino acid and Z for any amino acid, preferably a basic amino acid,
In some embodiments, where m is 0, and in case of an N-terminal oxidoreductase motif (the oxidoreductase motif is located at the N-terminal beginning of the immunogenic peptide), the first cysteine, threonine or serine of the motif can be chemically modified through N-acetylation, N-methylation, N-ethylation or N-propionylation.

In some embodiments, where m is 0, and in case of a C-terminal oxidoreductase motif (the oxidoreductase motif is located at the C-terminal end of the immunogenic peptide), the last cysteine, threonine or serine of the motif can be chemically modified through C-terminal substitution by acetyl, methyl, ethyl or propionyl groups of it's C-terminal amide or acid groups.
These motifs are further exemplified below.
In a preferred embodiment, said oxidoreductase motif can comprise the tetrapeptide sequence Cxx[CST] [SEQ ID NO: 1] or [CST]xxC [SEQ ID NO: 2], optionally preceded by one or more basic amino acids, such as HCXXC, KCXXC, RCXXC, KHCXXC, HKCXXC, RHCXXC, HRCXXC, KRCXXC, or RKCXXC.
In preferred embodiments, said oxidoreductase motif does not naturally occur within a region of 11 amino acids N- or C-terminally adjacent to the T-cell epitope, more preferably, said oxidoreductase motif does not naturally occur within the T-cell epitope.
In certain embodiments, the MHC class II T cell insulin epitope can be defined by sequence LALEGSLQK [SEQ ID NO: 3].
7. The method or use according to any one of aspects 1 to 6, wherein said peptide comprises the sequence Cxx[CST]SLQPLALEGSLQK [SEQ ID NO: 4] or [CST]xxCSLQPLALEGSLQK [SEQ ID NO:5].
8. The method or use according to any one of aspects 1 to 7, wherein said peptide comprises the sequence CxxCSLQPLALEGSLQK [SEQ ID NO: 6].
9. The method or use according to any one of aspects 1 to 8, wherein said peptide comprises the sequence HCxx[CST]SLQPLALEGSLQK [SEQ ID NO: 7] or H[CST]xxCSLQPLALEGSLQK [SEQ ID NO:8].
10. The method or use according to any one of aspects 1 to 9, wherein said peptide comprises the sequence HCxxCSLQPLALEGSLQK [SEQ ID NO: 9].
11. The method or use according to any one of aspects 1 to 10, wherein said peptide comprises the sequence Cxx[CST] [SEQ ID NO: 1] or [CST]xxC [SEQ ID NO: 2] redox motif sequence and the sequence SLQPLALEGSLQKRG [SEQ ID NO: 20].
12. The method or use according to any one of aspects 1 to 11, wherein said peptide comprises or consists of amino acid sequence HCPYCSLQPLALEGSLQKRG [SEQ ID NO: 26],
13. The method or use according to any one of aspects 1 to 12, wherein said peptide is administered as a pharmaceutical composition comprising said peptide and a pharmaceutically acceptable carrier.
14. The method or use according to any one of aspects 2 to 13, wherein said peptide is administered in a dosage regimen of between 50 and 1500 μg, preferably between 100 and 1200 μg.
15. The method or use according to any one of aspects 2 to 14, wherein said peptide is administered in a single dose, or in 2, 3, 4, 5, or more doses, either simultaneously or consecutively.
16. The method or use according to any one of aspects 2 to 15, wherein said peptide is administered through 4 bi-weekly subcutaneous or intramuscular injections according to any one of the following schemes:
   1) a first subcutaneous injection with 50 μg of said peptide, followed by three consecutive subcutaneous injections of 25 μg of said peptide, each performed 2 weeks apart;
   2) a first subcutaneous injection with 150 μg of said peptide, followed by three consecutive subcutaneous injections of 75 μg of said peptide, each performed 2 weeks apart; and
   3) a first subcutaneous injection with 450 μg of said peptide, followed by three consecutive subcutaneous injections of 225 μg of said peptide, each performed 2 weeks apart.
17. The method or use according to any one of aspects 2 to 16, wherein said patients are additionally HLA-DR3 negative (HLA-DR3−).
18. The method or use according to any one of aspects 2 to 17, wherein said peptide is administered as a pharmaceutical composition comprising said peptide and a pharmaceutically acceptable carrier.
19. The method or use according to any one of aspects 2 to 18, wherein said peptide is administered as a pharmaceutical composition comprising said peptide and an adjuvant.
   Other specific embodiments of the immunogenic peptides used in any one of the embodiments or aspects disclosed herein consist of any one of the following sequences:

[SEQ ID NO: 10]
Cxx[CST]SLQPLALEGSLQKRG,
[SEQ ID NO: 11]
[CST]xxCSLQPLALEGSLQKRG,
[SEQ ID NO: 12]
CxxCSLQPLALEGSLQKRG,
[SEQ ID NO: 13]
HCxx[CST]SLQPLALEGSLQKRG,
[SEQ ID NO: 14]
H[CST]xxCSLQPLALEGSLQKRG,
or
[SEQ ID NO: 15]
HCxxCSLQPLALEGSLQKRG.

In specific embodiments of such immunogenic peptide sequences, the Cxx[CST] [SEQ ID NO: 1] is CPY[CST] [SEQ ID NO: 16], and/or the [CST]xxC [SEQ ID NO: 2] is [CST]PYC [SEQ ID NO: 17], more specific CxxC [SEQ ID NO: 18] is CPYC [SEQ ID NO:19].
In a specific embodiment the peptide consists of the sequence HCPYCVRSLQPLALEGSLQKRG [SEQ ID. NO: 25] or HCPYCSLQPLALEGSLQKRG [SEQ ID NO: 26].
In any one of the above aspects or embodiments the redox motif is at the N terminal side of the epitope.
In an alternative set of aspects or embodiments the peptides have the redox motif at the C terminal side of the epitope.
20. Another aspect of the invention relates to any one of the peptides as disclosed above for use as a medicament, especially in the treatment or prevention of type 1 diabetes or for reducing the symptoms of type 1 diabetes, wherein the patient or subject has been determined as being positive for the DR4 HLA haplotype of the MHC class II molecules and optionally HLA-DR3 negative (HLA-DR3−).
   In some embodiments of said aspect 20, said haplotype determination in said patients is performed using polymerase chain reaction (PCR)-based analysis, sequence analysis, electrophoretic analysis or through antibody testing.
   In some embodiments of said aspect 20, patients homozygous for HLA type DR4+are deemed most responsive and/or patients heterozygous for HLA type DR4+such as patients being DR4+and DR3+are deemed moderately responsive.
21. Another aspect relates to pharmaceutical compositions comprising any one of the peptides as disclosed above and a pharmaceutically acceptable carrier, for use in the treatment or prevention of type 1 diabetes or for reducing the symptoms of type 1 diabetes, wherein the patient or subject has been determined as being positive for the DR4 HLA haplotype of the MHC class II molecules and optionally HLA-DR3 negative (HLA-DR3−).
22. In alternative embodiments, the patient or subject that is or has been determined as being positive for the DR4 HLA haplotype of the MHC class II molecules and optionally HLA-DR3 negative (HLA-DR3−) can be treated using a population of cytolytic CD4+ T cells, against APC presenting insulin epitopes obtained by the following in vitro method for the generation of a population of cytolytic CD4+ T cells, against APC presenting insulin epitopes, comprising the steps of:
   providing peripheral blood cells;
      contacting said cells in vitro with any one of the immunogenic peptides as disclosed above; and
      expanding said cells in the presence of IL-2.
23. Another aspect relates to a population of cells cytolytic CD4+ T cells, against insulin presenting APC obtainable by the above method for use in the treatment or prevention of type 1 diabetes or for reducing the symptoms of type 1 diabetes, wherein the patient or subject has been determined as being positive for the DR4 HLA haplotype of the MHC class II molecules and optionally HLA-DR3 negative (HLA-DR3−).
   In some embodiments of said aspect 22 or 23, said haplotype determination in said patients is performed using polymerase chain reaction (PCR)-based analysis, sequence analysis, electrophoretic analysis or through antibody testing.
   In some embodiments of said aspect 22 or 23, patients homozygous for HLA type DR4+ are deemed most responsive and/or patients heterozygous for HLA type DR4+ such as patients being DR4+ and DR3+ are deemed moderately responsive.

### BRIEF DESCRIPTION OF THE FIGURES

FIG. 1: represents the binding of the two tests peptides defined by the sequence HCPYCVRSLQPLALEGSLQKRG (SEQ ID NO: 25) and HCPYCSLQPLALEGSLQKRG (SEQ ID NO: 26) to DRB1*0301 (left panel) or DRB1*0401 (right panel) recombinant MHC II proteins. Test peptide binding is demonstrated dose-dependently by decreased fluorescence signal (RFU) due to competition with fluorescently tagged control high affinity binder peptide.

FIG. 2: represents the reactivity of different T1D patients measured by enumerating live responding CD4+ T cells after 1, 4 and 6 specific re-stimulations with an immunogenic peptide with the sequence HCPYCVRSLQPLALEGSLQKRG presented by autologous dendritic cells (SEQ ID NO: 25).

FIG. 3: represents scheme of phase Ib study design with the immunogenic peptide defined by the sequence HCPYCSLQPLALEGSLQKRG (SEQ ID NO: 26).

FIG. 4: represents boxplots of post-challenge 2 hours C-peptide AUC following a MMTT in different HLA-genotype subgroups. P1=Placebo, C1=Cohort 1, C2=Cohort 2, C3=Cohort 3. Data are expressed as percentage of variation of the response at 6 months post inclusion in the phase Ib study ((V8-V2)/V2).

FIG. 5: represents boxplots of the dose of insulin per Kg in different HLA-genotypes subgroups. P1=Placebo, C1=Cohort 1, C2=Cohort 2, C3=Cohort 3. Data are expressed as percentage of variation of the response at 6 months post inclusion in the phase Ib study ((V8-V2)/V2).

FIG. 6: represents boxplots of measured vs expected post-challenge C-peptide following 2 hours AUC of MMTT in different HLA-genotype subgroups. Pl=Placebo, C1=Cohort 1, C2=Cohort 2, C3=Cohort 3. Data are expressed as percentage of variation of the response (delta ratio) at 3 months (V6) and 6 months (V8) post inclusion in the phase Ib study.

FIG. 7: represents boxplots the dose of insulin per Kg in different HLA-genotypes subgroups across V3 to V8. Data are expressed as percentage of variation of the response ((Visit X-V2)/V2) in the phase Ib study.

### DETAILED DESCRIPTION OF THE INVENTION

The present invention will be described with respect to particular embodiments but the invention is not limited thereto but only by the claims. Any reference signs in the claims shall not be construed as limiting the scope. The following terms or definitions are provided solely to aid in the understanding of the invention. Unless specifically defined herein, all terms used herein have the same meaning as they would to one skilled in the art of the present invention. The definitions provided herein should not be construed to have a scope less than the one understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise. The term “any” when used in relation to aspects, claims or embodiments as used herein refers to any single one (i.e. anyone) as well as to all combinations of said aspects, claims or embodiments referred to.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. Said terms also encompass the embodiments “consisting essentially of” and “consisting of”.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

As used herein, the term “for use” as used in “composition for use in treatment of a disease” shall disclose also the corresponding method of treatment and the corresponding use of a preparation for the manufacture of a medicament for the treatment of a disease”.

The term “peptide” as used herein refers to a molecule comprising an amino acid sequence of between 12 and 200 amino acids, connected by peptide bonds, but which can comprise non-amino acid structures.

Peptides according to the invention can contain any of the conventional 20 amino acids or modified versions thereof, or can contain non-naturally occurring amino-acids incorporated by chemical peptide synthesis or by chemical or enzymatic modification.

The term “antigen” as used herein refers to a structure of a macromolecule, typically protein (with or without polysaccharides) or made of proteic composition comprising one or more hapten (s) and comprising T cell epitopes.

The term “antigenic protein” as used herein refers to a protein comprising one or more T cell epitopes. An auto-antigen or auto-antigenic protein as used herein refers to a human or animal protein present in the body, which elicits an immune response within the same human or animal body.

The term “epitope” refers to one or several portions (which may define a conformational epitope) of an antigenic protein which is/are specifically recognised and bound by an antibody or a portion thereof (Fab′, Fab2′, etc.) or a receptor presented at the cell surface of a B or T cell lymphocyte, and which is able, by said binding, to induce an immune response.

The term “T cell epitope” in the context of the present invention refers to a dominant, sub-dominant or minor T cell epitope, i.e. a part of an antigenic protein that is specifically recognised and bound, when complexed with a MHC class II molecule, by a receptor expressed at the cell surface of a T lymphocyte. Whether an epitope is dominant, sub-dominant or minor depends on the immune reaction elicited against the epitope. Dominance depends on the frequency at which such epitopes are recognised by T cells and able to activate them, among all the possible T cell epitopes of a protein.

The T cell epitope is an epitope that is recognised and associates to MHC class II molecules, which consists of a sequence of +/−9 amino acids which fit in the groove of the MHC II molecule. Within a peptide sequence representing a T cell epitope, the amino acids in the epitope are numbered P1 to P9, amino acids N-terminal of the epitope are numbered P-−1, P-−2 and so on, amino acids C terminal of the epitope are numbered P+1, P+2 and so on. Peptides recognised by MHC class II molecules and not by MHC class I molecules are referred to as MHC class II restricted T cell epitopes.

The term “MHC” refers to “major histocompatibility antigen”. In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such the terms “MHC” and “HLA” are equivalents when used herein. The HLA system in man has its equivalent in the mouse, i.e., the H2 system. The most intensely-studied HLA genes are the nine so-called classical MHC genes: HLA-A, HLA-B, HLA-C, HLA-DPA1, HLA-DPB1, HLA-DQA1, HLAs DQB1, HLA-DRA, and HLA-DRB1. In humans, the MHC is divided into three regions: Class I, II, and III. The A, B, and C genes belong to MHC class I, whereas the six D genes belong to class II. MHC class I molecules are made of a single polymorphic chain containing 3 domains (alpha 1, 2 and 3), which associates with beta 2 microglobulin at cell surface. Class II molecules are made of 2 polymorphic chains, each containing 2 chains (alpha 1 and 2, and beta 1 and 2).

Class I MHC molecules are expressed on virtually all nucleated cells.

Peptide fragments presented in the context of class I MHC molecules are recognised by CD8+ T lymphocytes (cytolytic T lymphocytes or CTLs). CD8+ T lymphocytes frequently mature into cytolytic effectors which can lyse cells bearing the stimulating antigen. Class II MHC molecules are expressed primarily on activated lymphocytes and antigen-presenting cells. CD4+ T lymphocytes (helper T lymphocytes or Th) are activated with recognition of a unique peptide fragment presented by a class II MHC molecule, usually found on an antigen-presenting cell like a macrophage or dendritic cell. CD4+ T lymphocytes proliferate and secrete cytokines such as IL-2, IFN-gamma and IL-4 that support antibody-mediated and cell mediated responses. Functional HLAs are characterised by a deep binding groove to which endogenous as well as foreign, potentially antigenic peptides bind. The groove is further characterised by a well-defined shape and physico-chemical properties. HLA class I binding sites are closed, in that the peptide termini are pinned down into the ends of the groove. They are also involved in a network of hydrogen bonds with conserved HLA residues. In view of these restraints, the length of bound peptides is limited to 8, 9 or 10 residues. However, it has been demonstrated that peptides of up to 12 amino acid residues are also capable of binding HLA class I. Comparison of the structures of different HLA complexes confirmed a general mode of binding wherein peptides adopt a relatively linear, extended conformation, or can involve central residues to bulge out of the groove.

In contrast to HLA class I binding sites, class II sites are open at both ends. This allows peptides to extend from the actual region of binding, thereby “hanging out” at both ends. Class II HLAs can therefore bind peptide ligands of variable length, ranging from 9 to more than 25 amino acid residues. Similar to HLA class I, the affinity of a class II ligand is determined by a “constant” and a “variable” component. The constant part again results from a network of hydrogen bonds formed between conserved residues in the HLA class II groove and the main-chain of a bound peptide. However, this hydrogen bond pattern is not confined to the N-and C-terminal residues of the peptide but distributed over the whole chain. The latter is important because it restricts the conformation of complexed peptides to a strictly linear mode of binding. This is common for all class II allotypes. The second component determining the binding affinity of a peptide is variable due to certain positions of polymorphism within class II binding sites. Different allotypes form different complementary pockets within the groove, thereby accounting for subtype-dependent selection of peptides, or specificity. Importantly, the constraints on the amino acid residues held within class II pockets are in general “softer” than for class I. There is much more cross reactivity of peptides among different HLA class II allotypes. The sequence of the +/−9 amino acids (i.e. 8, 9 or 10) of an MHC class II T cell epitope that fit in the groove of the MHC II molecule are usually numbered P1 to P9. Additional amino acids N-terminal of the epitope are numbered P-−1, P-−2 and so on, amino acids C-terminal of the epitope are numbered P+1, P+2 and so on.

At the genetic level, the MHC class II cluster is located on the short arm of chromosome 6 (6p21). The cluster includes three classical class II genes (HLA-DP, -DQ and DR) and two non-classical class II genes (HLA-DM and -DO). The structure of MHC class II is achieved by the association of two membrane bound chains, called □ and □, that create the antigen-binding cleft of MHC class II. Both □ and □ chains are encoded by distinct loci closely linked as pairs of □ and □ genes, i.e. DR□/DR□, DQ□/DQ□ and DP□/DP□ HLA-DP, -DQ and DR loci are highly polymorphic, especially in the antigen-binding pocket of the class II molecule. HLA-DP and -DQ contain polymorphisms in both the -□and -□ chain genes (DPA, DPB, DQA and DQB). In HLA-DR, polymorphism concerns only the DR □ chain (DRB gene). There are 9 DRB loci (numbered from DRB1 to DRB9), but only the DRB1 locus is found on all haplotypes, and hence constitutes the major determinant of classical DR serology (McCluskey et al, Current Protocols in Immunology (2017), 118, A.1S.1-A.1S.6).

Taking as an example the HLA-DRB1 group, literature has reported the existence of over 40 different haplotypes (Marsh et al, Tissue Antigens (2010), 75, p291). Of most relevance throughout the human population are the DRB1*03 and DRB1*04 haplotype groups. In the DRB1*03 group, two alleles are common, namely DRB1*0301 and DRB1*0302, but other alleles have been reported, such as DRB1*0303, DRB1*0304 and DRB1*0307. In the DRB1*04 group, ten major alleles can be found, namely: DRB1*0401, DRB1*0402, DRB1*0403, DRB1*0404, DRB1*0405, DRB1*0406, DRB1*0407, DRB1*0408, DRB1*0410 and DRB1*0411. The term “DR4 positive” or “DR4+” used throughout the application indicates that the subject is positive for one of the DRB1*04 haplotypes Similarly, the term “DR3 positive” or “DR3+” used throughout the application indicates that the subject is positive for one of the DRB1*03 haplotypes. The term “DR4 negative” or “DR4−” used throughout the application indicates that the subject does not have any of the DRB1*04 haplotypes Similarly, the term “DR3 negative” or “DR3−” used throughout the application indicates that the subject does not have any of the DRB1*03 haplotypes.

HLA typing can be performed using techniques known in the art including, without limitation, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing, solid-phase sequencing, sequencing with mass spectrometry such as matrix-assisted laser desorption/ionization time-of-flight mass spectrometry, and sequencing by hybridization. Non-limiting examples of electrophoretic analysis include lab gel electrophoresis such as agarose or polyacrylamide gel electrophoresis, capillary electrophoresis, and denaturing gradient gel electrophoresis. Other methods for genotyping an individual at a polymorphic site in a marker include, e.g., the INVADER® assay from Third Wave Technologies, Inc., restriction fragment length polymorphism (RFLP) analysis, allele-specific oligonucleotide hybridization, a heteroduplex mobility assay, and single strand conformational polymorphism (SSCP) analysis.

Alternatively, HLA typing can be performed by antibody testing.

The term “homologue” as used herein with reference to the epitopes used in the context of the invention, refers to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular homologues of an epitope correspond to the natural epitope modified in at most three, more particularly in at most 2, most particularly in one amino acid.

The term “derivative” as used herein with reference to the peptides of the invention refers to molecules which contain at least the peptide active portion (i.e. the redox motif and the MHC class II epitope capable of eliciting cytolytic CD4+ T cell activity) and, in addition thereto comprises a complementary portion which can have different purposes such as stabilising the peptides or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. In particular, the sequence identity is from 70% to 80%, from 81% to 85%, from 86% to 90%, from 91% to 95%, from 96% to 100%, or 100%.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. The nucleic acid encoding a peptide according to the invention or fragment thereof is a sequence encoding the peptide or fragment thereof originating from a mammal or corresponding to a mammalian, most particularly a human peptide fragment.

The term “immune disorders” or “immune diseases” refers to diseases wherein a reaction of the immune system is responsible for or sustains a malfunction or non-physiological situation in an organism. Included in immune disorders are, inter alia, allergic disorders and autoimmune diseases.

The terms “autoimmune disease” or “autoimmune disorder” refer to diseases that result from an aberrant immune response of an organism against its own cells and tissues due to a failure of the organism to recognise its own constituent parts (down to the sub-molecular level) as “self”. The group of diseases can be divided in two categories, organ-specific and systemic diseases. An “allergen” is defined as a substance, usually a macromolecule or a proteic composition which elicits the production of lgE antibodies in predisposed, particularly genetically disposed, individuals (atopics) patients Similar definitions are presented in Liebers et al. (1996) Clin. Exp. Allergy 26, 494-516.

The term “type 1 diabetes” (T1D) or “diabetes type 1” (also known as “type 1 diabetes mellitus” or “immune mediated diabetes” or formerly known as “juvenile onset diabetes” or “insulin dependent diabetes”) is an autoimmune disorder that typically develops in susceptible individuals during childhood. At the basis of T1D pathogenesis is the destruction of most insulin-producing pancreatic beta-cells by an autoimmune mechanism. In short, the organism loses the immune tolerance towards the pancreatic beta-cells in charge of insulin production and induces an immune response, mainly cell-mediated, associated to the production of autoantibodies, which leads to the self-destruction of beta-cells.

The term “therapeutically effective amount” refers to an amount of the peptide of the invention or derivative thereof, which produces the desired therapeutic or preventive effect in a patient. For example, in reference to a disease or disorder, it is the amount which reduces to some extent one or more symptoms of the disease or disorder, and more particularly returns to normal, either partially or completely, the physiological or biochemical parameters associated with or causative of the disease or disorder. Typically, the therapeutically effective amount is the amount of the peptide of the invention or derivative thereof, which will lead to an improvement or restoration of the normal physiological situation. For instance, when used to therapeutically treat a mammal affected by an immune disorder, it is a daily amount peptide/kg body weight of the said mammal Alternatively, where the administration is through gene-therapy, the amount of naked DNA or viral vectors is adjusted to ensure the local production of the relevant dosage of the peptide of the invention, derivative or homologue thereof.

The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence.

Amino acids are referred to herein with their full name, their three-letter abbreviation or their one letter abbreviation.

Motifs of amino acid sequences are written herein according to the format of Prosite. Motifs are used to describe a certain sequence variety at specific parts of a sequence. The symbol X is used for a position where any amino acid is accepted. Alternatives are indicated by listing the acceptable amino acids for a given position, between square brackets (‘[]’). For example: [CST] stands for an amino acid selected from Cys, Ser or Thr Amino acids which are excluded as alternatives are indicated by listing them between curly brackets (‘{}’). For example {AM} stands for any amino acid except Ala and Met. The different elements in a motif are optionally separated from each other by a hyphen (-). Repetition of an identical element within a motif can be indicated by placing behind that element a numerical value or a numerical range between parentheses. For example X(2) corresponds to X-X or XX; X(2, 5) corresponds to 2, 3, 4 or 5 X amino acids, A(3) corresponds to A-A-A or AAA.

To distinguish between the amino acids X, those between H and C are called external amino acids X (single underlined in the above sequence), those within the redox motif are called internal amino acids X (double underlined in the above sequence).

X represents any amino acid, particularly an L-amino acid, more particularly one of the 20 naturally occurring L-amino acids.

A peptide, comprising a T cell epitope and a modified peptide motif sequence, having reducing activity is capable of generating a population of antigen-specific cytolytic CD4+ T cell towards antigen-presenting cells.

Accordingly, in its broadest sense, the invention relates to the use of peptides which comprise at least one T-cell epitope of an antigen (self or non-self) with a potential to trigger an immune reaction, and an “oxidoreductase”, “thioreductase” “thioredox”, or “redox” (all terms can be used interchangeable herein) sequence motif with a reducing activity on peptide disulfide bonds. The MHC class II T cell epitope and the modified redox motif sequence may be immediately adjacent to each other in the peptide or optionally separated by a one or more amino acids (so called linker sequence). Optionally the peptide additionally comprises an endosome targeting sequence and/or additional “flanking” sequences.

The peptides disclosed herein comprise an MHC class II T-cell epitope of an insulin antigen with a potential to trigger an immune reaction, and a modified redox motif. The reducing activity of the motif sequence in the peptide can be assayed for its ability to reduce a sulfhydryl group such as in the insulin solubility assay wherein the solubility of insulin is altered upon reduction, or with a fluorescence-labelled substrate such as insulin. An example of such assay uses a fluorescent peptide and is described in Tomazzolli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

The (modified) redox motif may be positioned at the amino-terminus side of the T-cell epitope or at the carboxy-terminus of the T-cell epitope.

Peptide fragments with reducing activity are encountered in thioreductases which are small disulfide reducing enzymes including glutaredoxins, nucleoredoxins, thioredoxins and other thiol/disulfide oxidoreductases (Holmgren (2000) Antioxid. Redox Signal. 2, 811-820; Jacquot et al. (2002) Biochem. Pharm. 64, 1065-1069). They are multifunctional, ubiquitous and found in many prokaryotes and eukaryotes. They exert reducing activity for disulfide bonds on proteins (such as enzymes) through redox active cysteines within conserved active domain consensus sequences: CXXC [SEQ ID NO:18], CXXS [SEQ ID NO:23], CXXT [SEQ ID NO:24], SXXC

[SEQ ID NO:21], TXXC [SEQ ID NO:22] (Fomenko et al. (2003) Biochemistry 42, 11214-11225; Fomenko et al. (2002) Prot. Science 11, 2285-2296), in which X stands for any amino acid. Such domains are also found in larger proteins such as protein disulfide isomerase (PDI) and phosphoinositide-specific phospholipase C. The 4 amino acid redox motif as known from e.g. Fomenko and WO2008/017517 comprises a cysteine at position 1 and/or 4; thus the motif is either CXX[CST] [SEQ ID NO: 1] or [CST]XXC [SEQ ID NO:2]. Such a tetrapeptide sequence will be referred to as “the motif”. The motif in a peptide can be any of the alternatives CXXC [SEQ ID NO:18], SXXC [SEQ ID NO:21], TXXC [SEQ ID NO:22], CXXS [SEQ ID NO:23] or CXXT [SEQ ID NO:24]. In particular, peptides contain the sequence motif CXXC [SEQ ID NO:18].

As explained in detail further on, the peptides used in the present invention can be made by chemical synthesis, which allows the incorporation of non-natural amino acids. Accordingly, “C” in the above recited redox modified redox motifs represents either cysteine or another amino acid with a thiol group such as mercaptovaline, homocysteine or other natural or non-natural amino acids with a thiol function. In order to have reducing activity, the cysteines present in a modified redox motif should not occur as part of a cystine disulfide bridge. Nevertheless, a redox modified redox motif may comprise modified cysteines such as methylated cysteine, which is converted into cysteine with free thiol groups in vivo. X can be any of the 20 natural amino acids, including S, C, or T or can be a non-natural amino acid. In particular embodiments X is an amino acid with a small side chain such as Gly, Ala, Ser or Thr. In further particular embodiments, X is not an amino acid with a bulky side chain such as Trp. In further particular embodiments X is not Cysteine. In further particular embodiments at least one X in the modified redox motif is His. In other further particular embodiments at least one X in the modified redox is Pro.

Peptides may further comprise modifications to increase stability or solubility, such as modification of the N-terminal NH₂ group or the C terminal COOH group (e.g. modification of the COOH into a CONH₂ group).

The terms “oxidoreductase motif”, “thiol-oxidoreductase motif”, “thioreductase motif”, “thioredox motif ” or “redox motif” are used herein as synonymous terms and refers to motifs involved in the transfer of electrons from one molecule (the reductant, also called the hydrogen or electron donor) to another (the oxidant, also called the hydrogen or electron acceptor).

In particular, the term “oxidoreductase motif” can refer to the known [CST]XXC or CXX[CST] motifs, but particularly refers to the more general sequence motif

Zm[CST]XnC or ZmCXn[CST],

wherein n is an integer from 0 to 6, such as 0, 1, 2, 3, 4, 5 or 6.

wherein m stands for an integer from 0 to 2, such as 0, 1 or 2.

in which C stands for cysteine, S for serine, T for threonine, X for any amino acid and

Z for any amino acid, preferably a basic amino acid

In order to have reducing activity, the cysteines present in a modified oxidoreductase motif should not occur as part of a cystine disulfide bridge.

Typically, the oxidoreductase motif can comprise the general formula ZmCXnC,

wherein X is any amino acid,

Z is a basic amino acid preferably selected from H, K or R, and

wherein n is an integer from 0 to 3 and

m is an integer from 0 to 2.

The term “basic amino acid” refers to any amino acid that acts like a Bronsted-Lowry and Lewis base, and includes natural basic amino acids such as Arginine (R), Lysine (K) or Histidine (H), or non-natural basic amino acids, such as, but not limited to:
lysine variants like Fmoc-β-Lys(Boc)-OH (CAS Number 219967-68-7), Fmoc-Orn(Boc)-OH also called L-ornithine or ornithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)-OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

Hence, in addition to the commonly known thioredox motif CXXC and its variants disclosed herein, there are also motifs wherein the two cysteine moieties are adjacent to each other (CC) or wherein the two cysteine moieties are separated by 1, 3, 4, 5, or 6 amino acids such as CXC, CXXXC, CXXXXC, CXXXXXC or CXXXXXXC. In any one of said embodiments, one of the cysteines can also be changed into an S or T.

Typically one or more basic amino acid “Z” such as selected from H, K, or R, can be added to the thioredox motif such as H-motif, K-motif, R-motif, KH-motif, HK-motif, RH-motif, HR-motif, KR-motif, or RK-motif.

Particularly interesting examples of thioredox motifs that can be used in the present invention are:

CC, HCC, KCC, RCC;

CXC, HCXC, KCXC, RCXC, KHCXC, HKCXC, RHCXC, HRCXC, RKCXC, KRCXC;

CXXC, HCXXC, KCXXC, RCXXC, KHCXXC, HKCXXC, RHCXXC, HRCXXC, RKCXXC, KRCXXC;

CXXXC, HCXXXC, KCXXXC, RCXXXC, KHCXXXC, HKCXXXC, RHCXXXC, HRCXXXC, RKCXXXC, KRCXXXC; CXXXC, HCXXXC, KCXXXC, RCXXXC, KHCXXXC, HKCXXXC, RHCXXXC,

HRCXXXC, RKCXXXC, KRCXXXC; CXXXXC, HCXXXXC, KCXXXXC, RCXXXXC, KHCXXXXC, HKCXXXXC, RHCXXXXC, HRCXXXXC, RKCXXXXC, KRCXXXXC;

CXXXXXC, HCXXXXXC, KCXXXXXC, RCXXXXXC, KHCXXXXXC, HKCXXXXXXXC, RHCXXXXXC, HRCXXXXXC, RKCXXXXXC, KRCXXXXXC;

CXXXXXXC, HCXXXXXXC, KCXXXXXXC, RCXXXXXXC, KHCXXXXXXC, HKCXXXXXXC, RHCXXXXXXC, HRCXXXXXXC, RKCXXXXXXC, KRCXXXXXXC;

Specific examples of the CXC motif are: CHC, CKC, CRC, CGC, CAC, CVC, CLC, CIC, CMC, CFC, CWC, CPC, CSC, CTC, CYC, CNC, CQC, CDC, and CEC. Any one of these exemplary CXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein. Preferred examples of such motifs are: KCHC, KCKC, KCRC, KCGC, KCAC, KCVC, KCLC, KCIC, KCMC, KCFC, KCWC, KCPC, KCSC, KCTC, KCYC, KCNC, KCQC, KCDC, KCEC, HCHC, HCKC, HCRC, HCGC, HCAC, HCVC, HCLC, HCIC, HCMC, HCFC, HCWC, HCPC, HCSC, HCTC, HCYC, HCNC, HCQC, HCDC, HCEC, RCHC, RCKC, RCRC, RCGC, RCAC, RCVC, RCLC, RCIC, RCMC, RCFC, RCWC, RCPC, RCSC, RCTC, RCYC, RCNC, RCQC, RCDC, and RCEC;

In a preferred embodiment, said oxidoreductase motif is CX₃C, i.e. CXXXC, typically CX¹X²X³C, wherein X¹, X², and X³, each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², and X³ in said motif is any amino acid except for C, S, or T. In a specific embodiment, at least one of X¹, X², or X³ in said motif is a basic amino acid, such as H, K, or R, or a non-natural basic amino acid as defined herein.

Specific examples of the CXXXC motif are: CXPYC, CPXYC, and CPYXC, wherein X can be can be any amino acid, more preferably CXPYC, such as: CKPYC, CRPYC (SEQ ID NO: 55), CHPYC, CGPYC, CAPYC, CVPYC, CLPYC, CIPYC, CMPYC, CFPYC, CWPYC, CPPYC, CSPYC, CTPYC, CCPYC, CYPYC, CNPYC, CQPYC, CDPYC, and CEPYC; or

CPXYC, such as: CPKYC, CPRYC, CPHYC, CPGYC, CPAYC, CPVYC, CPLYC, CPIYC, CPMYC, CPFYC, CPWYC, CPPYC, CPSYC, CPTYC, CPCYC, CPYYC, CPNYC, CPQYC, CPDYC, CPEYC, and CPLYC; or

CPYXC, such as: CPYKC, CPYRC, CPYHC, CPYGC, CPYAC, CPYVC, CPYLC, CPYIC, CPYMC, CPYFC, CPYWC, CPYPC, CPYSC, CPYTC, CPYCC, CPYYC, CPYNC, CPYQC, CPYDC, CPYEC, and CPYLC.

Further specific examples of the CXXXC motif are: CXHGC, CHXGC, and CHGXC, wherein X can be can be any amino acid, more preferably CXHGC, such as: CKHGC, CRHGC, CHHGC, CGHGC, CAHGC, CVHGC, CLHGC, CIHGC, CMHGC, CFHGC, CWHGC, CPHGC, CSHGC, CTHGC, CCHGC, CYHGC, CNHGC, CQHGC, CDHGC, CEHGC, and CKHGC; or

CGXHC, such as: CGKHC, CGRHC, CGHHC, CGGHC, CGAHC, CGVHC, CGLHC, CGIHC, CGMHC, CGFHC, CGWHC, CGPHC, CGSHC, CGTHC, CGCHC, CGYHC, CGNHC, CGQHC, CGDHC, CGEHC, and CGLHC; or

CHGXC, such as: CHGKC, CHGRC, CHGHC, CHGGC, CHGAC, CHGVC, CHGLC, CHGIC, CHGMC, CHGFC, CHGWC, CHGPC, CHGSC, CHGTC, CHGCC, CHGYC, CHGNC, CHGQC, CHGDC, CHGEC, and CHGLC.

Further specific examples of the CXXXC motif are: CXGPC, CGXPC, and CGPXC, wherein X can be can be any amino acid, more preferably CXGPC, such as: CKGPC, CRGPC, CHGPC, CGGPC, CAGPC, CVGPC, CLGPC, CIGPC, CMGPC, CFGPC, CWGPC, CPGPC, CSGPC, CTGPC, CCGPC, CYGPC, CNGPC, CQGPC, CDGPC, CEGPC, and CKGPC; or

CGXPC, such as: CGKPC, CGRPC, CGHPC, CGGPC, CGAPC, CGVPC, CGLPC, CGIPC, CGMPC, CGFPC, CGWPC, CGPPC, CGSPC, CGTPC, CGCPC, CGYPC, CGNPC, CGQPC, CGDPC, CGEPC, and CGLPC; or

CGPXC, such as: CGPKC, CGPRC, CGPHC, CGPGC, CGPAC, CGPVC, CGPLC, CGPIC, CGPMC, CGPFC, CGPWC, CGPPC, CGPSC, CGPTC, CGPCC, CGPYC, CGPNC, CGPQC, CGPDC, CGPEC, and CGPLC.

Further specific examples of the CXXXC motif are: CXGHC, CGXHC, and CGHXC, wherein X can be can be any amino acid, more preferably CXGHC, such as: CKGHC, CRGHC, CHGHC, CGGHC, CAGHC, CVGHC, CLGHC, CIGHC, CMGHC, CFGHC, CWGHC, CPGHC, CSGHC, CTGHC, CCGHC, CYGHC, CNGHC, CQGHC, CDGHC, CEGHC, and CKGHC; or

CGXFC, such as: CGKFC, CGRFC, CGHFC, CGGFC, CGAFC, CGVFC, CGLFC, CGIFC, CGMFC, CGFFC, CGWFC, CGPFC, CGSFC, CGTFC, CGCFC, CGYFC, CGNFC, CGQFC, CGDFC, CGEFC, and CGLFC; or

CGHXC, such as: CGHKC, CGHRC, CGHHC, CGHGC, CGHAC, CGHVC, CGHLC, CGHIC, CGHMC, CGHFC, CGHWC, CGHPC, CGHSC, CGHTC, CGHCC, CGHYC, CGHNC, CGHQC, CGHDC, CGHEC, and CGHLC.

Further specific examples of the CXXXC motif are: CXGFC, CGXFC, and CGFXC, wherein X can be can be any amino acid, more preferably CXGFC, such as: CKGFC, CRGFC, CHGFC, CGGFC, CAGFC, CVGFC, CLGFC, CIGFC, CMGFC, CFGFC, CWGFC, CPGFC, CSGFC, CTGFC, CCGFC, CYGFC, CNGFC, CQGFC, CDGFC, CEGFC, and CKGFC; or

CGXFC, such as: CGKFC, CGRFC, CGHFC, CGGFC, CGAFC, CGVFC, CGLFC, CGIFC, CGMFC, CGFFC, CGWFC, CGPFC, CGSFC, CGTFC, CGCFC, CGYFC, CGNFC, CGQFC, CGDFC, CGEFC, and CGLFC; or

CGFXC, such as: CGFKC, CGFRC, CGFHC, CGFGC, CGFAC, CGFVC, CGFLC, CGFIC, CGFMC, CGFFC, CGFWC, CGFPC, CGFSC, CGFTC, CGFCC, CGFYC, CGFNC, CGFQC, CGFDC, CGFEC, and CGFLC.

Further specific examples of the CXXXC motif are: CXRLC, CRXLC, and CRLXC, wherein X can be can be any amino acid, more preferably CXRLC, such as: CKRLC, CRRLC, CHRLC, CGRLC, CARLC, CVRLC, CLRLC, CIRLC, CMRLC, CFRLC, CWRLC, CPRLC, CSRLC, CTRLC, CCRLC, CYRLC, CNRLC, CQRLC, CDRLC, CERLC, and CKRLC; or

CRXLC, such as: CRKLC, CRRLC, CRHLC, CRGLC, CRALC, CRVLC, CRLLC, CRILC, CRMLC, CRFLC, CRWLC, CRPLC, CRSLC, CRTLC, CRCLC, CRYLC, CRNLC, CRQLC, CRDLC, CRELC, and CRLLC; or

CRLXC, such as: CRLKC, CRLRC, CRLHC, CRLGC, CRLAC, CRLVC, CRLLC, CRLIC, CRLMC, CRLFC, CRLWC, CRLPC, CRLSC, CRLTC, CRLCC, CRLYC, CRLNC, CRLQC, CRLDC, CRLEC, and CRLLC.

Further specific examples of the CXXXC motif are: CXHPC, CHXPC, and CHPXC, wherein X can be can be any amino acid, more preferably CXHPC, such as: CKHPC, CRHPC, CHHPC, CGHPC, CAHPC, CVHPC, CLHPC, CIHPC, CMHPC, CFHPC, CWHPC, CPHPC, CSHPC, CTHPC, CCHPC, CYHPC, CNHPC, CQHPC, CDHPC, CEHPC, and CKHPC; or

CHXPC, such as: CHKPC, CHRPC, CHHPC, CHGPC, CHAPC, CHVPC, CHLPC, CHIPC, CHMPC, CHFPC, CHWPC, CHPPC, CHSPC, CHTPC, CHCPC, CHYPC, CHNPC, CHQPC, CHDPC, CHEPC, and CHLPC; or

CHPXC, such as: CHPKC, CHPRC, CHPHC, CHPGC, CHPAC, CHPVC, CHPLC, CHPIC, CHPMC, CHPFC, CHPWC, CHPPC, CHPSC, CHPTC, CHPCC, CHPYC, CHPNC, CHPQC, CHPDC, CHPEC, and CHPLC.

Any one of these exemplary CXXXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

In a preferred embodiment, said oxidoreductase motif is CX₄C, i.e. CXXXXC, typically CX¹X²X³X⁴C, wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³ and X⁴ in said motif is any amino acid except for C, S, or T. In a specific embodiment, at least one of X¹, X², X³ or X⁴ in said motif is a basic amino acid, such as H, K, or R, or a non-natural basic amino acid as defined herein.

Specific examples of the CXXXXC motif are: CLAVLC, CTVQAC or CGAVHC and their variants such as: CX¹AVLC, CLX²VLC, CLAX³LC, or CLAVX⁴C; CX¹VQAC, CTX²QAC, CTVX³AC, or CTVQX⁴C; CX¹AVHC, CGX²VHC, CGAX³HC, or CGAVX⁴C; wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein.

Any one of these exemplary CXXXXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

In a preferred embodiment, said oxidoreductase motif is CX₅C, i.e. CXXXXXC, typically CX¹X²X³X⁴X⁵C, wherein X¹, X², X³, X⁴ and X⁵ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³, X⁴ and X⁵ in said motif is any amino acid except for C, S, or T. In a specific embodiment, at least one of X¹, X², X³ X⁴ or X⁵ in said motif is a basic amino acid, such as H, K, or R, or a non-natural basic amino acid as defined herein.

Specific examples of the CXXXXXC motif are: CPAFPLC or CDQGGEC and their variants such as: CX¹AFPLC, CPX²FPLC, CPAX³PLC, CPAFX⁴LC, or CPAFPX⁵C; CX¹QGGEC, CDX²GGEC, CDQX³GEC, CDQGX⁴EC, or CDQGGX⁵C, wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural basic amino acids as defined herein. Any one of these exemplary CXXXXXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

In a preferred embodiment, said oxidoreductase motif is CX₆C, i.e. CXXXXXXC, typically CX¹X²X³X⁴X⁵X⁶C, wherein X¹, X², X³, X⁴X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³, X⁴, X⁵ and X⁶ in said motif is any amino acid except for C, S, or T. In a specific embodiment, at least one of X¹, X², X³ X⁴, X⁵ or X⁶ in said motif is a basic amino acid, such as H, K, or R, or a non-natural basic amino acid as defined herein.

A specific example of the CXXXXXXC motif is: CDIADKYC or variants thereof such as: CX¹IADKYC, CDX²ADKYC, CDIX³DKYC, CDIAX⁴KYC, CDIADX⁵YC, or CDIADKX⁶C, wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural basic amino acids as defined herein.

Any one of these exemplary CXXXXXXC motifs can be preceded by one or more amino acids (Zₘ), wherein m is an integer between 0 and 3, preferably 0 or 1, and wherein Z is any amino acid, preferably a basic amino acid, such as H, K, or R, or a non-natural basic amino acid as defined herein.

Particularly preferred examples of such oxidoreductase motifs are:

C[KHR]C, CX[KHR]XC, CXX[KHR]C, C[KHR]XXC, [KHR]CC, [KHR]CXC, [KHR]XXXC CC[KHR], CXC[KHR], CXXXC[KHR], [KHR]CC[KHR], [KHR]CXC[KHR], [KHR]CXXXC[KHR], [KHR]C[KHR]C, C[KHR]C[KHR], [KHR]CXX[KHR]C, [KHR]CX[KHR]XC, [KHR]C[KHR]XXC, CXX[KHR]C[KHR], CX[KHR]XC[KHR], C[KHR]XXC[KHR], and the like.

In any one of the motif embodiments herein, if m is 0 and in case of an N-terminal oxidoreductase motif (the oxidoreductase motif is located at the N-terminal beginning of the immunogenic peptide), the first cysteine, threonine or serine of the motif can be chemically modified through N-acetylation, N-methylation, N-ethylation or N-propionylation.

In any one of the motif embodiments herein, if m is 0 and in case of a C-terminal oxidoreductase motif (the oxidoreductase motif is located at the C-terminal end of the immunogenic peptide), the last cysteine, threonine or serine of the motif can be chemically modified through C-terminal substitution by acetyl, methyl, ethyl or propionyl groups of it's C-terminal amide or acid groups.

In the peptides used in the present invention comprising a modified redox motif, the motif is located such that, when the epitope fits into the MHC groove, the motif remains outside of the MHC binding groove. The modified redox motif is placed either immediately adjacent to the epitope sequence within the peptide [in other words a linker sequence of zero amino acids between motif and epitope], or is separated from the T cell epitope by a linker comprising an amino acid sequence of 7 amino acids or less. More particularly, the linker comprises 1, 2, 3, 4,5, 6 or 7 amino acids. Specific embodiments are peptides with a 0, 12, 3 or 4 amino acid linker between epitope sequence and modified redox motif sequence. Preferably the linker comprises an amino acid sequence of 4 amino acids. In those peptides where the modified redox motif sequence is adjacent to the epitope sequence this is indicated as position P−4 to P−1 or P+1 to P+4 compared to the epitope sequence. Apart from a peptide linker, other organic compounds can be used as linker to link the parts of the peptide to each other (e.g. the modified redox motif sequence to the T cell epitope sequence).

The peptides used in the present invention can further comprise additional short amino acid sequences N or C-terminally of the sequence comprising the T cell epitope and the modified redox motif. Such an amino acid sequence is generally referred to herein as a ‘flanking sequence’. A flanking sequence can be positioned between the epitope and an endosomal targeting sequence and/or between the modified redox motif and an endosomal targeting sequence. In certain peptides, not comprising an endosomal targeting sequence, a short amino acid sequence may be present N and/or C terminally of the modified redox motif and/or epitope sequence in the peptide. More particularly a flanking sequence is a sequence of between 1 and 7 amino acids, most particularly a sequence of 2 amino acids.

The modified redox motif may be located N-terminal from the epitope.

In certain embodiments of the present invention, peptides used are provided comprising one epitope sequence and a modified redox motif sequence. In further particular embodiments, the modified redox motif occurs several times (1, 2, 3, 4 or even more times) in the peptide, for example as repeats of the modified redox motif which can be spaced from each other by one or more amino acids or as repeats which are immediately adjacent to each other. Alternatively, one or more modified redox motifs are provided at both the N and the C terminus of the T cell epitope sequence.

Other variations envisaged for the peptides of the present invention include peptides which contain repeats of a T cell epitope sequence wherein each epitope sequence is preceded and/or followed by the modified redox motif (e.g. repeats of “modified redox motif-epitope” or repeats of “modified redox motif-epitope-modified redox motif”). Herein the modified redox motifs can all have the same sequence but this is not obligatory. It is noted that repetitive sequences of peptides which comprise an epitope which in itself comprises the modified redox motif will also result in a sequence comprising both the ‘epitope’ and a ‘modified redox motif’. In such peptides, the modified redox motif within one epitope sequence functions as a modified redox motif outside a second epitope sequence.

Typically the peptides used in the present invention comprise only one T cell epitope. As described below a T cell epitope in a protein sequence can be identified by functional assays and/or one or more in silica prediction assays. The amino acids in a T cell epitope sequence are numbered according to their position in the binding groove of the MHC proteins. A T-cell epitope present within a peptide consist of between 8 and 25 amino acids, yet more particularly of between 8 and 16 amino acids, yet most particularly consists of 8, 9, 10, 11, 12, 13, 14, 15 or 16 amino acids.

In a more particular embodiment, the T cell epitope consists of a sequence of 9 amino acids. In a further particular embodiment, the T-cell epitope is an epitope, which is presented to T cells by MHC-class II molecules [MHC class II restricted T cell epitopes]. Typically T cell epitope sequence refers to the octapeptide or more specifically nonapeptide sequence which fits into the cleft of an MHC II protein.

The T cell epitope of the peptides of the present invention can correspond either to a natural epitope sequence of a protein or can be a modified version thereof, provided the modified T cell epitope retains its ability to bind within the MHC cleft, similar to the natural T cell epitope sequence. The modified T cell epitope can have the same binding affinity for the MHC protein as the natural epitope, but can also have a lowered affinity. In particular, the binding affinity of the modified peptide is no less than 10-fold less than the original peptide, more particularly no less than 5 times less. Peptides of the present invention have a stabilising effect on protein complexes. Accordingly, the stabilising effect of the peptide-MHC complex compensates for the lowered affinity of the modified epitope for the MHC molecule.

The sequence comprising the T cell epitope and the reducing compound within the peptide can be further linked to an amino acid sequence (or another organic compound) that facilitates uptake of the peptide into late endosomes for processing and presentation within MHC class II determinants. The late endosome targeting is mediated by signals present in the cytoplasmic tail of proteins and correspond to well-identified peptide motifs. The late endosome targeting sequences allow for processing and efficient presentation of the antigen-derived T cell epitope by MHC-class II molecules. Such endosomal targeting sequences are contained, for example, within the gp75 protein (Vijayasaradhi et al. (1995) J. Cell. Biol. 130, 807-820), the human CD3 gamma protein, the HLA-BM 11 (Copier et al. (1996) J. Immunol. 157, 1017-1027), the cytoplasmic tail of the DEC205 receptor (Mahnke et al. (2000) J. Cell Biol. 151, 673-683). Other examples of peptides which function as sorting signals to the endosome are disclosed in the review of Bonifacio and Traub (2003) Annu. Rev. Biochem. 72, 395-447. Alternatively, the sequence can be that of a subdominant or minor T cell epitope from a protein, which facilitates uptake in late endosome without overcoming the T cell response towards the antigen. The late endosome targeting sequence can be located either at the amino-terminal or at the carboxy-terminal end of the antigen derived peptide for efficient uptake and processing and can also be coupled through a flanking sequence, such as a peptide sequence of up to 10 amino acids. When using a minor T cell epitope for targeting purpose, the latter is typically located at the amino-terminal end of the antigen derived peptide.

Accordingly, the present invention envisages the use of peptides of antigenic proteins and their use in eliciting specific immune reactions. These peptides can either correspond to fragments of proteins which comprise, within their sequence i.e. a reducing compound and a T cell epitope separated by at most 10, preferably 7 amino acids or less. Alternatively, and for most antigenic proteins, the peptides of the invention are generated by coupling a reducing compound, more particularly a reducing modified redox motif as described herein, N-terminally or C-terminally to a T cell epitope of the antigenic protein (either directly adjacent thereto or with a linker of at most 10, more particularly at most 7 amino acids). Moreover the T cell epitope sequence of the protein and/or the modified redox motif can be modified and/or one or more flanking sequences and/or a targeting sequence can be introduced (or modified), compared to the naturally occurring sequence. Thus, depending on whether or not the features of the present invention can be found within the sequence of the antigenic protein of interest, the peptides of the present invention can comprise a sequence which is ‘artificial’ or ‘naturally occurring’.

The peptides of the present invention can vary substantially in length. The length of the peptides can vary from 13 or 14 amino acids, i.e. consisting of an epitope of 8-9 amino acids, adjacent thereto the modified redox motif 5 amino acids with the histidine, up to 20, 25, 30, 40 or 50 amino acids. For example, a peptide may comprise an endosomal targeting sequence of 40 amino acids, a flanking sequence of about 2 amino acids, a motif as described herein of 5 amino acids, a linker of 4 amino acids and a T cell epitope peptide of 9 amino acids.

Accordingly, in particular embodiments, the complete peptide consists of between 13 amino acids up 20, 25, 30, 40, 50, 75 or 100 amino acids. More particularly, where the reducing compound is a modified redox motif as described herein, the length of the (artificial or natural) sequence comprising the epitope and modified redox motif optionally connected by a linker (referred to herein as ‘epitope-modified redox motif’ sequence), without the endosomal targeting sequence, is critical. The ‘epitope-modified redox motif’ more particularly has a length of 13, 14, 15, 16, 17, 18 or 19 amino acids. Such peptides of 13 or 14 to 19 amino acids can optionally be coupled to an endosomal targeting signal of which the size is less critical.

As detailed above, in particular embodiments, the peptides of the present invention comprise a reducing modified redox motif as described herein linked to a T cell epitope sequence.

In further particular embodiments, the peptides used in the invention are peptides comprising T cell epitopes which do not comprise an amino acid sequence with redox properties within their natural sequence.

However, in alternative embodiments, the T cell epitope may comprise any sequence of amino acids ensuring the binding of the epitope to the MHC cleft. Where an epitope of interest of an antigenic protein comprises a modified redox motif such as described herein within its epitope sequence, the immunogenic peptides according to the present invention comprise the sequence of a modified redox motif as described herein and/or of another reducing sequence coupled N- or C-terminally to the epitope sequence such that (contrary to the modified redox motif present within the epitope, which is buried within the cleft) the attached modified redox motif can ensure the reducing activity.

Accordingly the T cell epitope and motif are immediately adjacent or separated from each other and do not overlap. To assess the concept of “immediately adjacent” or “separated”, the 8 or 9 amino acid sequence which fits in the MHC cleft is determined and the distance between this octapeptide or nonapeptide with the redox motif tetrapeptide or modified redox motif pentapeptide including histidine is determined.

Generally, the peptides used in the present invention are not natural (thus no fragments of proteins as such) but artificial peptides which contain, in addition to a T cell epitope, a modified redox motif as described herein, whereby the modified redox motif is immediately separated from the T cell epitope by a linker consisting of up to seven, most particularly up to four or up to 2 amino acids.

It has been shown that upon administration (i.e. injection) to a mammal of a peptide disclosed herein (or a composition comprising such a peptide), the peptide elicits the activation of T cells recognising the antigen derived T cell epitope and provides an additional signal to the T cell through reduction of surface receptor. This supra-optimal activation results in T cells acquiring cytolytic properties for the cell presenting the T cell epitope, as well as suppressive properties on bystander T cells. In this way, the peptides or composition comprising the peptides described in the present invention, which contain an antigen-derived T cell epitope and, outside the epitope, a modified redox motif can be used for direct immunisation of mammals, including human beings. The invention thus provides the use of peptides disclosed herein or derivatives thereof, for use as a medicine. Accordingly, the present invention provides therapeutic methods which comprise administering one or more peptides disclosed herein to a patient in need thereof.

The present invention offers methods by which antigen-specific T cells endowed with cytolytic properties can be elicited by immunisation with small peptides. It has been found that peptides which contain (i) a sequence encoding a T cell epitope from an antigen and (ii) a consensus sequence with redox properties, and further optionally also comprising a sequence to facilitate the uptake of the peptide into late endosomes for efficient MHC-class II presentation, elicit suppressor T-cells.

The immunogenic properties of the disclosed peptides are of particular interest in the treatment and prevention of immune reactions.

Peptides described herein are used as medicament, more particularly used for the manufacture of a medicament for the prevention or treatment of an immune disorder in a mammal, more in particular in a human

The present invention describes methods of treatment or prevention of an immune disorder of a mammal in need for such treatment or prevention, by using the peptides disclosed herein, homologues or derivatives thereof, the methods comprising the step of administering to said mammal suffering or at risk of an immune disorder a therapeutically effective amount of the peptides disclosed herein, homologues or derivatives thereof such as to reduce the symptoms of the immune disorder. The treatment of both humans and animals, such as, pets and farm animals is envisaged. In an embodiment the mammal to be treated is a human. The immune disorders referred to above are in a particular embodiment selected from allergic diseases and autoimmune diseases.

The peptides for use in the invention or the pharmaceutical composition comprising such peptides as defined herein is preferably administered through sub-cutaneous or intramuscular administration. Preferably, the peptides or pharmaceutical compositions comprising such can be injected sub-cutaneously (SC) in the region of the lateral part of the upper arm, midway between the elbow and the shoulder. When two or more separate injections are needed, they can be administered concomitantly in both arms.

The peptide for use in the invention or the pharmaceutical composition comprising such is administered in a therapeutically effective dose. Exemplary but non-limiting dosage regimens are between 50 and 1500 μg, preferably between 100 and 1200 μg. More specific dosage schemes can be between 50 and 250 μg, between 250 and 450 μg or between 850 and 1300 μg, depending on the condition of the patient and severity of disease. Dosage regimen can comprise the administration in a single dose or in 2, 3, 4, 5, or more doses, either simultaneously or consecutively. Exemplary non-limiting administration schemes are the following:
A low dose scheme comprising the SC administration of 50 μg of peptide in two separate injections of 25 μg each (100 μL each) followed by three consecutive injections of 25 μg of peptide as two separate injections of 12.5 μg each (50 μL each).
A medium dose scheme comprising the SC administration of 150 μg of peptide in two separate injections of 75 μg each (300 μL each) followed by three consecutive administrations of 75 μg of peptide as two separate injections of 37.5 μg each (150 μL each).
A high dose scheme comprising the SC administration of 450 μg of peptide in two separate injections of 225 μg each (900 μL each) followed by three consecutive administrations of 225 μg of peptide as two separate injections of 112.5 μg each (450 μL each).

For all the above peptides additional variant are envisaged, wherein between Histidine and Cysteine, one or two amino acids X are present. Typically these external amino acid(s) X is (are) not His, Cys, Ser or Thr.

The peptides for use in the present invention can also be used in diagnostic in vitro methods for detecting class II restricted CD4+ T cells in a sample. In this method a sample is contacted with a complex of an MHC class II molecule and a peptide disclosed herein. The CD4+ T cells detected by measuring the binding of the complex with cells in the sample, wherein the binding of the complex to a cell is indicative for the presence of CD4 +T cells in the sample.

The complex can be a fusion protein of the peptide and an MHC class II molecule. Alternatively MHC molecules in the complex are tetramers. The complex can be provided as a soluble molecule or can be attached to a carrier.

Accordingly, in particular embodiments, the methods of treatment and prevention of the present invention comprise the administration of an immunogenic peptide as described herein, wherein the peptide comprise a T cell epitope of an antigenic protein which plays a role in the disease to be treated (for instance such as those described above). In further particular embodiments, the epitope used is a dominant epitope, combined with method of stratification or selection of those patients that are assumed to benefit the most of said treatment.

Peptides for use in accordance with the present invention can be prepared by synthesising a peptide wherein T cell epitope and modified redox motif will be separated by 0 to 5 amino acids. In certain embodiments the modified redox motif can be obtained by introducing 1, 2 or 3 mutations outside the epitope sequence, to preserve the sequence context as occurring in the protein. Typically amino-acids in P-−2 and P-−1, as well as in P+10 and P+11, with reference to the nonapeptide which are part of the natural sequence are preserved in the peptide sequence. These flanking residues generally stabilize the binding to MHC class II. In other embodiments the sequence N terminal or C terminal of the epitope will be unrelated to the sequence of the antigenic protein containing the T cell epitope sequence.

Thus based upon the above methods for designing a peptide, a peptide is generated by chemical peptide synthesis, recombinant expression methods or in more exceptional cases, proteolytic or chemical fragmentation of proteins.

Peptides as produced in the above methods can be tested for the presence of a T cell epitope in in vitro and in vivo methods, and can be tested for their reducing activity in in vitro assays. As a final quality control, the peptides can be tested in in vitro assays to verify whether the peptides can generate CD4+ T cells which are cytolytic via an apoptotic pathway for antigen presenting cells presenting the antigen which contains the epitope sequence which is also present in the peptide with the modified redox motif.

The peptides for use in the present invention can be generated using recombinant DNA techniques, in bacteria, yeast, insect cells, plant cells or mammalian cells. In view of the limited length of the peptides, they can be prepared by chemical peptide synthesis, wherein peptides are prepared by coupling the different amino acids to each other. Chemical synthesis is particularly suitable for the inclusion of e.g. D-amino acids, amino acids with non-naturally occurring side chains or natural amino acids with modified side chains such as methylated cysteine. Chemical peptide synthesis methods are well described and peptides can be ordered from companies such as Applied Biosystems and other companies.

Peptide synthesis can be performed as either solid phase peptide synthesis (SPPS) or contrary to solution phase peptide synthesis. The best known SPPS methods are t-Boc and Fmoc solid phase chemistry:

During peptide synthesis several protecting groups are used. For example hydroxyl and carboxyl functionalities are protected by t-butyl group, lysine and tryptophan are protected by t-Boc group, and asparagine, glutamine, cysteine and histidine are protected by trityl group, and arginine is protected by the pbf group. If appropriate, such protecting groups can be left on the peptide after synthesis. Peptides can be linked to each other to form longer peptides using a ligation strategy (chemoselective coupling of two unprotected peptide fragments) as originally described by Kent (Schnelzer & Kent (1992) Int J. Pept. Protein Res. 40, 180-193) and reviewed for example in Tam et al. (2001) Biopolymers 60, 194-205 provides the tremendous potential to achieve protein synthesis which is beyond the scope of SPPS. Many proteins with the size of 100-300 residues have been synthesised successfully by this method. Synthetic peptides have continued to play an ever increasing crucial role in the research fields of biochemistry, pharmacology, neurobiology, enzymology and molecular biology because of the enormous advances in the SPPS.

Alternatively, the peptides can be synthesised by using nucleic acid molecules which encode the peptides of this invention in an appropriate expression vector which include the encoding nucleotide sequences. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridisation methodologies. Such DNA molecules may be incorporated into expression vectors, including plasmids, which are adapted for the expression of the DNA and production of the polypeptide in a suitable host such as bacterium, e.g. Escherichia coli, yeast cell, animal cell or plant cell.

The physical and chemical properties of a peptide of interest (e.g. solubility, stability) are examined to determine whether the peptide is/would be suitable for use in therapeutic compositions. Typically this is optimised by adjusting the sequence of the peptide. Optionally, the peptide can be modified after synthesis (chemical modifications e.g. adding/deleting functional groups) using techniques known in the art.

T cell epitopes on their own are thought to trigger early events at the level of the T helper cell by binding to an appropriate HLA molecule on the surface of an antigen presenting cell and stimulating the relevant T cell subpopulation. These events lead to T cell proliferation, lymphokine secretion, local inflammatory reactions, the recruitment of additional immune cells to the site, and activation of the B cell cascade leading to production of antibodies. One isotype of these antibodies, lgE, is fundamentally important in the development of allergic symptoms and its production is influenced early in the cascade of events, at the level of the T helper cell, by the nature of the lymphokines secreted. A T cell epitope is the basic element or smallest unit of recognition by a T cell receptor where the epitope comprises amino acid residues essential to receptor recognition, which are contiguous in the amino acid sequence of the protein.

However, upon administration of the peptides with a T-cell epitope and a redox motif, the following events are believed to happen:

activation of antigen (i) specific T cells resulting from cognate interaction with the antigen-derived peptide presented by MHC-class II molecules;

the reductase sequence reduces T cell surface proteins, such as the CD4 molecule, the second domain of which contains a constrained disulfide bridge. This transduces a signal into T cells.

Among a series of consequences related to increased oxidative pathway, important events are increased calcium influx and translocation of the NF-kB transcription factor to the nucleus. The latter results in increased transcription of IFN-gamma and granzymes, which allows cells to acquire cytolytic properties via an apoptosis-inducing mechanism; the cytolytic property affects cells presenting the peptide by a mechanism, which involves granzyme B secretion, and Fas-FasL interactions. Since the cell killing effect is obtained via an apoptotic pathway, cytolytic cells is a more appropriate term for these cells than cytotoxic cells. Destruction of the antigen-presenting target cells prevents activation of other T cells specific for epitopes located on the same antigen, or to an unrelated antigen that would be processed by the same antigen-presenting cell; an additional consequence of T cell activation is to suppress activation of bystander T cells by a cell-cell contact dependent mechanism. In such a case, T cells activated by an antigen presented by a different antigen-presenting cell is also suppressed provided both cytolytic and bystander T cells are in close proximity, namely activated on the surface of the same antigen-presenting cell.

The above-postulated mechanism of action is substantiated with experimental data disclosed in the above cited PCT application WO2008/017517.

The present invention provides methods for generating antigen-specific cytolytic CD4+ T cells either in vivo or in vitro and their use in treating patients that have been stratified or selected as benefiting the most of said treatment. Independently thereof, methods to discriminate cytolytic CD4+ T cells from other cell populations such as Foxp3+ Tregs based on characteristic expression data can be envisaged.

The present invention describes in vivo methods for the production of the antigen-specific CD4+ T cells that can be used for treatment in light of the present invention. A particular embodiment relates to the method for producing or isolating the CD4+ T cells by immunising animals (including humans) with the peptides as described herein and then isolating the CD4+ T cells from the immunised animals. The present invention describes in vitro methods for the production of antigen specific cytolytic CD4+ T cells towards APC. The present application also discloses methods for generating antigen specific cytolytic CD4+ T cells towards APC.

In one embodiment, methods are provided which comprise the isolation of peripheral blood cells, the stimulation of the cell population in vitro by an immunogenic peptide described herein and the expansion of the stimulated cell population, more particularly in the presence of IL-2. The methods according to the invention have the advantage a high number of CD4+ T cells is produced and that the CD4+ T cells can be generated which are specific for the antigenic protein (by using a peptide comprising an antigen-specific epitope).

In an alternative embodiment, the CD4+ T cells can be generated in vivo, i.e. by the injection of the immunogenic peptides described herein to a subject, and collection of the cytolytic CD4+ T cells generated in vivo.

The antigen-specific cytolytic CD4+ T cells towards APC, obtainable by the methods disclosed herein are of particular interest for the administration to mammals for immunotherapy, in the prevention of allergic reactions and the treatment of auto-immune diseases. Both the use of allogenic and autogeneic cells are envisaged.

Cytolytic CD4+ T cells populations are obtained as described herein below.

Antigen-specific cytolytic CD4+ T cells as described herein can be used as a medicament, more particularly for use in adoptive cell therapy, more particularly in the treatment of acute allergic reactions and relapses of autoimmune diseases such as multiple sclerosis. Isolated cytolytic CD4+ T cells or cell populations, more particularly antigen-specific cytolytic CD4+ T cell populations generated as described are used for the manufacture of a medicament for the prevention or treatment of immune disorders. Methods of treatment by using the isolated or generated cytolytic CD4+ T cells are disclosed.

As explained in WO2008/017517 cytolytic CD4+ T cells towards APC can be distinguished from natural Treg cells based on expression characteristics of the cells. More particularly, a cytolytic CD4+ T cell population demonstrates one or more of the following characteristics compared to a natural Treg cell population:

an increased expression of surface markers including CD103, CTLA-4, Fasl and ICOS upon activation,

intermediate expression of CD25,

expression of CD4, ICOS, CTLA-4, GITR and low or no expression of CD127 (IL7-R), no expression of CD27.

expression of transcription factor T-bet and egr-2 (Krox-20) but not of the transcription repressor Foxp3,

a high production of IFN-gamma and no or only trace amounts of IL-10, IL-4, IL-5, IL-13 or TGF-beta.

Further the cytolytic T cells express CD45RO and/or CD45RA, do not express CCR7, CD27 and present high levels of granzyme B and other granzymes as well as Fas ligand.

The peptides for use in the invention will, upon administration to a living animal, typically a human being, elicit specific T cells exerting a suppressive activity on bystander T cells.

In specific embodiments the cytolytic cell populations disclosed herein are characterised by the expression of FasL and/or Interferon gamma. In specific embodiments the cytolytic cell populations of the present invention are further characterised by the expression of GranzymeB.

This mechanism also implies and the experimental results show that the peptides of the invention, although comprising a specific T-cell epitope of a certain antigen, can be used for the prevention or treatment of disorders elicited by an immune reaction against other T-cell epitopes of the same antigen or in certain circumstances even for the treatment of disorders elicited by an immune reaction against other T-cell epitopes of other different antigens if they would be presented through the same mechanism by MHC class II molecules in the vicinity of T cells activated by peptides of the invention.

Isolated cell populations of the cell type having the characteristics described above, which, in addition are antigen-specific, i.e. capable of suppressing an antigen-specific immune response are disclosed.

The present invention provides the use of pharmaceutical compositions comprising one or more peptides according to the present invention, further comprising a pharmaceutically acceptable carrier. As detailed above, the present invention also relates to the compositions for use as a medicine or to methods of treating a mammal of an immune disorder by using the composition and to the use of the compositions for the manufacture of a medicament for the prevention or treatment of immune disorders, combined with method of stratification or selection of those patients that are assumed to benefit the most of said treatment. The pharmaceutical composition could for example be a vaccine suitable for treating or preventing immune disorders, especially airborne and foodborne allergy, as well as diseases of allergic origin. As an example described further herein of a pharmaceutical composition, a peptide according to the invention is adsorbed on an adjuvant suitable for administration to mammals, such as aluminium hydroxide (alum). Typically, the desired dosage as described herein, such as 50 μg to 1500 μg of the peptide, adsorbed on alum, are injected by the subcutaneous route on 3 occasions at an interval of 2 weeks. It should be obvious for those skilled in the art that other routes of administration are possible, including oral, intranasal or intramuscular. Also, the number of injections and the amount injected can vary depending on the conditions to be treated. Further, other adjuvants than alum can be used, provided they facilitate peptide presentation in MHC-class II presentation and T cell activation. Thus, while it is possible for the active ingredients to be administered alone, they typically are presented as pharmaceutical formulations. The formulations, both for veterinary and for human use, of the present invention comprise at least one active ingredient, as above described, together with one or more pharmaceutically acceptable carriers. The present disclosure relates to pharmaceutical compositions, comprising, as an active ingredient, one or more peptides described herein, in admixture with a pharmaceutically acceptable carrier. The pharmaceutical composition should comprise a therapeutically effective amount of the active ingredient, such as indicated hereinafter in respect to the method of treatment or prevention. Optionally, the composition further comprises other therapeutic ingredients. Suitable other therapeutic ingredients, as well as their usual dosage depending on the class to which they belong, are well known to those skilled in the art and can be selected from other known drugs used to treat immune disorders.

The term “pharmaceutically acceptable carrier” as used herein means any material or substance with which the active ingredient is formulated in order to facilitate its application or dissemination to the locus to be treated, for instance by dissolving, dispersing or diffusing the composition, and/or to facilitate its storage, transport or handling without impairing its effectiveness. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the immunogenic peptide in the composition. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders. Suitable pharmaceutical carriers for use in the pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives typically contain 8 to 22 carbon atoms. Examples of alkylarylsulphonates are the sodium, calcium or alcanolamine salts of dodecyl benzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidyl-ethanolamine, phosphatidylserine, phosphatidylglycerine, lysolecithin, cardio lipin, dioctanylphosphatidylcholine, dipalmitoylphoshatidylcholine and their mixtures. Suitable non-ionic surfactants include polyethoxylated and poly propoxylated derivatives of alkyl phenols, fatty alcohols, fatty acids, aliphatic amines or amides containing at least 12 carbon atoms in the molecule, alkylarene sulphonates and dialkylsulphosuccinates, such as polyglycol ether derivatives of aliphatic and cycloaliphatic alcohols, saturated and unsaturated fatty acids and alkylphenols, the derivatives typically containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants. Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, New Jersey, 1981), “Tensid-Taschenbucw’, 2 d ed. (Hanser Verlag, Vienna, 1981) and “Encyclopaedia of Surfactants, (Chemical Publishing Co., New York, 1981). Peptides, homologues or derivatives thereof according to the invention (and their physiologically acceptable salts or pharmaceutical compositions all included in the term “active ingredients”) may be administered by any route appropriate to the condition to be treated and appropriate for the compounds, here the proteins and fragments to be administered. Possible routes include regional, systemic, oral (solid form or inhalation), rectal, nasal, topical (including ocular, buccal and sublingual), vaginal and parenteral (including subcutaneous, intramuscular, intravenous, intradermal, intra-arterial, intrathecal and epidural). The preferred route of administration may vary with for example the condition of the recipient or with the diseases to be treated. As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Typical unit dosage formulations are those containing a daily dose or unit daily sub-dose, as herein above recited, or an appropriate fraction thereof, of an active ingredient. It should be understood that in addition to the ingredients particularly mentioned above the formulations of this invention may include other agents conventional in the art having regard to the type of formulation in question, for example those suitable for oral administration may include flavouring agents. Peptides, homologues or derivatives thereof according to the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polyniethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injection include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof. In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

Cytolytic CD4+ T cells as obtained as described herein, induce APC apoptosis after MHC-class II dependent cognate activation, affecting both dendritic and B cells, as demonstrated in vitro and in vivo, and (2) suppress bystander T cells by a contact-dependent mechanism in the absence of IL-10 and/or TGF-beta. Cytolytic CD4+ T cells can be distinguished from both natural and adaptive Tregs, as discussed in detail in WO2008/017517.

The present invention will now be illustrated by means of the following examples which are provided without any limiting intention. Furthermore, all references described herein are explicitly included herein by reference.

### EXAMPLES

### Example 1

### Binding of Immunogenic Peptides Comprising an Insulin MHCII T Cell Epitope and an Oxidoreductase Motif to Soluble DRB1*0301 or DRB1*0401 Recombinant MHC II Proteins

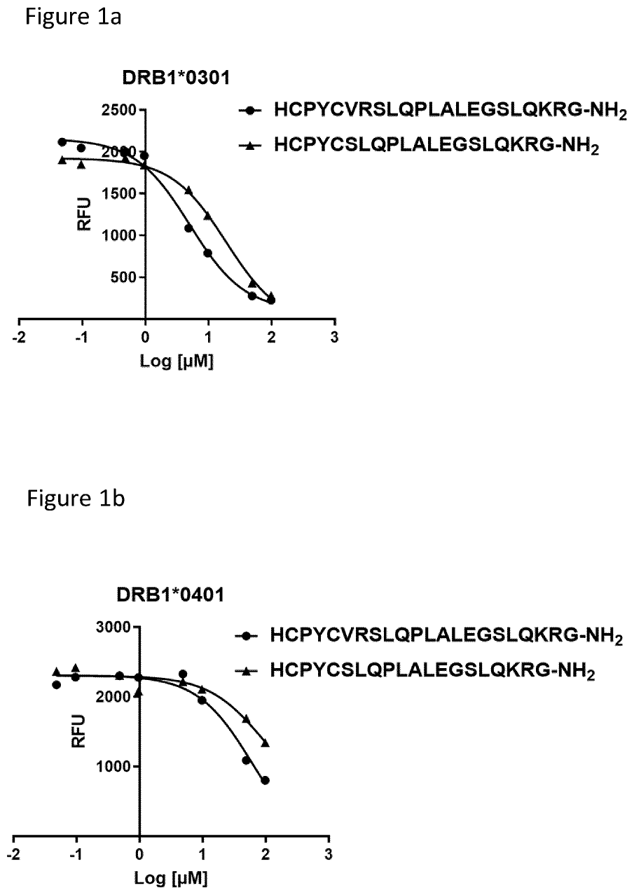

To test the binding of peptides comprising an MHC class II T cell epitope from proinsulin region C20_A1 and an oxidoreductase motif, a soluble phase competition assay was performed where increasing concentration of peptides with the sequence HCPYCVRSLQPLALEGSLQKRG (SEQ ID. NO: 25) and HCPYCSLQPLALEGSLQKRG (SEQ ID. NO: 26) compete with a labelled control peptide (high affinity binder; biotinylated) for binding to soluble DRB1*0301 or DRB1*0401 recombinant human MHC II proteins. As binding approaches equilibrium (18h), peptide-MHC II complexes are captured and separated from unbound reagents. Captured peptide-MHCII complexes are quantitatively detected by time-resolved fluorescence (Eu³⁺streptavidin) and data processed and plotted to ascertain dose-dependent binding properties of test peptide and determination of IC50 (decrease in fluorescence intensity reflects binding of the peptide). All the tests with these peptides were performed in triplicates, and each test was conducted two times. FIG. 1 shows the results of one experiment. Peptides with the sequence HCPYCVRSLQPLALEGSLQKRG (SEQ ID. NO: 25) and HCPYCSLQPLALEGSLQKRG (SEQ ID. NO: 26) are good binders for DRB1*0301 and DRB1*0401 haplotypes as they were able to compete with high affinity reference epitopes binders used in the assay.

### Example 2

### Capacity of an Immunogenic Peptides Comprising an Insulin MHCII T Cell Epitope and an Oxidoreductase Motif to Prime and Expand CD4+ T Cells From Different Insulin-Dependent Diabetes Mellitus Patients

Naive CD4+ T cells from different type 1 diabetes (T1D) patients were tested for their reactivity to the peptide defined by the sequence HCPYCVRSLQPLALEGSLQKRG. HLA DRB1 typing of T1D patient was first tested (see table 1).

TABLE 1
HLA DRB1 typing of tested T1D patients.

| Patient # | HLA typing | |
|---|---|---|
| T1D02 | DRB1*03 | DRB1*13 |
| T1D03 | DRB1*03 | DRB1*04 |
| T1D04 | DRB1*04 | DRB1*13 |
| T1D05 | DRB1*03 | DRB1*04 |
| T1D06 | DRB1*03 | DRB1*03 |
| T1D07 | DRB1*04 | DRB1*04 |
| T1D08 | DRB1*03 | DRB1*04 |

HLA typing was done using methods known in the art, such as for example reported in Mack et al., 2009, Tissue Antigens. 2009 Jan; 73(1): 17-32.

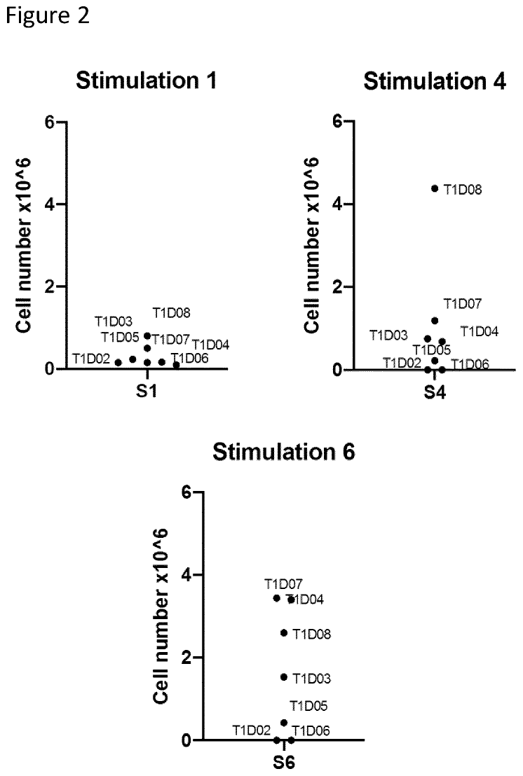

Blood samples were then processed and naive CD4+ T cells were purified by magnetic isolation techniques. The peptide capacity to prime and expand these naive CD4+ T cells was tested using peptide pre-loaded autologous dendritic cells (monocyte-derived DC differentiated in the presence of GM-CSF and IL-4 followed by maturation with TNF-α) as antigen presenting cells. FIG. 2 shows reactivity measured by evolution of CD4+ T cells number after specific peptide stimulation with autologous dendritic cells over successive restimulations (S1, S4 and S6). Data show good responsiveness (cell maintenance and amplification) for most of the patients tested except for two patients for which cell reactivity was not markedly observed and was associated with mortality already at stimulation 2 (T1D02 & T1D06; n cells=0 from S2 to S6). The data show that patients not expressing the DRB1*04 haplotype showed no marked response to peptide stimulation under said treatment test conditions. More strikingly, these non-responsive T1D patients expressed the DRB1*03 haplotype, for which the peptide showed good binding capacities in a competitive HLA binding assay.

### Example 3

### Phase Ib Clinical Trial in T1D Patients With an Immunogenic Peptide With the Sequence HCPYCSLQPLALEGSLQKRG

The safety, clinical efficiency and induced immune responses of an immunogenic peptide with the sequence HCPYCSLQPLALEGSLQKRG were evaluated in a phase Ib clinical trial in recent-onset (≤6 months) adult T1D patients. In this dose escalation, placebo-controlled study, patients received 4 bi-weekly subcutaneous injections of one of the 3 tested doses or matching placebo. The peptide was injected with alum as adjuvant. Patients were followed up for 6 months to evaluate the safety of the peptide and induced immune responses. The main inclusion criteria were:
Males and females 18 to 30 years of age BMI 17 28 kg/m²;
Initial diagnosis of Type 1 Diabetes according to ADA/WHO criteria within the past 6 months;
Insulin requirement, as determined by the investigator;
HLA-DR3 positive and/or HLA-DR4 positive;
Presence of at least one autoantibody (GAD65, IA 2 or ZnT8);
Fasting C peptide at screening ≥0 2 nmol/L and/or stimulated C peptide ≥0 4 nmol/L.

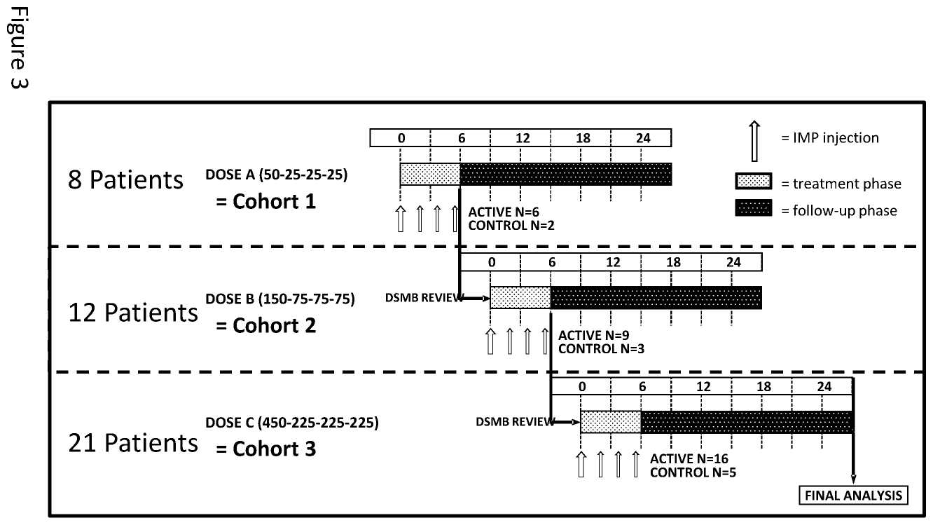

FIG. 3 shows the scheme of phase Ib study design. Patients were divided into 3 cohorts:
The low dose cohort (cohort 1) comprised 8 patients (6 treated and 2 placebo) injected SC with 50 μg of peptide followed by three consecutive injections of 25 μg of peptide. The 4 injections were performed 2 weeks apart. Patients were then followed up until week 24.
The medium dose (cohort 2) comprised 12 patients (9 treated and 3 placebo) injected SC with 150 μg of peptide followed by three consecutive administrations of 75 μg of peptide. The 4 injections were performed 2 weeks apart. Patients were then followed up until week 24.
The higher dose (cohort 3) comprised 21 patients (16 treated and 5 placebo) injected SC with 450 μg of peptide followed by three consecutive administrations of 225 μg of peptide. The 4 injections were performed 2 weeks apart. Patients were then followed up until week 24.

A datamining analysis, using systematic analysis of all data relations with the proprietary KEM® (Knowledge Extraction and Management) artificial intelligence technology from Ariana Pharma, was performed on the full data set from the clinical trial. This approach aimed to identify subgroups of patients with trends of improved clinical parameters. During this analysis, HLA genotype came up as a key element to consider when assessing future clinical response. It appears from the initial datamining results, in cohort 3 (the higher dose tested), that multiple parameters at multiple time points were improving in patients with an HLA-DR4 (+) genotype as well as in patients with an HLA-DR3 (−) genotype. Of importance and reinforcing this finding, patients with an HLA-DR4(−) genotype showed no improvement of the same parameters at the different time points under these phase Ib test conditions. These initial findings are summarized in the Table 2 below.

TABLE 2
Identification of subgroups in Cohort 3 with differential clinical parameter
evolution by data mining using data driven analysis with KEM ®.

| Study Arm | Subgroup | Endpoint | Visit | improvement |
|---|---|---|---|---|
| Cohort 3 | HLA_DR3{−} | CpeptideGlucose | 4 | yes |
| Cohort 3 | HLA_DR3{−} | MeasuredVsExpected_normAUC | 6 | yes |
| Cohort 3 | HLA_DR4{−} | CpeptideGlucose | 6 | no |
| Cohort 3 | HLA_DR4{−} | MeasuredVsExpected_normAUC | 6 | no |
| Cohort 3 | HLA_DR4{+} | MeasuredVsExpected_normAUC | 6 | yes |
| Cohort 3 | HLA_DR3{−} | MeasuredVsExpected_normAUC | 8 | Yes |
| Cohort 3 | HLA_DR4{+} | MeasuredVsExpected_normAUC | 8 | Yes |

DNA Isolation has been conducted according to the analytical plan provided by IMGM, by using the Chemagic STAR DNA Blood Kit (Chemagen) for the Hamilton Robot, and eluted into 150 μl Tris-HCL (pH 8.0). The Low resolution HLA Typing has been conducted according to the LABType® SSO Method sequence-specific oligonucleotide probes LABType SSO using sequence-specific oligonucleotide (SSO) probes bound to fluorescently coded microspheres to identify alleles encoded by the sample DNA. LABType applies Luminex® technology to the reverse SSO DNA typing method. (www.onelambda.com/en/product/labtype-sso.html) and evaluated with the HLA Fusion™ Software. For the High resolution HLA Typing a so called long Range PCR according to SOP AA-1550 has been used and this has been sequenced with the Illumina Technology. The evaluation of these sequences was conducted with the GenDX NGSengine Version 2.13.0 (www.gendx.com/products/ngsengine).

Based on this initial hypothesis-free driven finding, the clinical parameters were explored within different subpopulations of the clinical trial according to HLA-genotypes and more precisely expression of HLA-DR3 and HLA-DR4 haplotypes. Table 3 summarizes the distribution of the different genotypes in the trial. As it can be observed, the different groups (placebo, cohort 1, cohort 2 and cohort 3) are not balanced for the different genotype combinations. This imbalance is purely due to the small size of the study.

TABLE 3
number of patients in each arm of the study according
to their HLA-DR genotypes. X represents an HLA-DR
genotype other than DR3 or DR4.

| Study arm | DR4-X | DR4-DR4 | DR4-DR3 | DR3-DR3 | DR3-X | Total |
|---|---|---|---|---|---|---|
| Placebo | 0 | 2 | 6 | 0 | 2 | 10 |
| Cohort 1 | 1 | 2 | 1 | 1 | 1 | 6 |
| Cohort 2 | 5 | 1 | 2 | 1 | 0 | 9 |
| Cohort 3 | 7 | 1 | 3 | 1 | 4 | 16 |
| Total | 13 | 6 | 12 | 3 | 7 | 41 |

| | |
|---|---|
| DR3− (19) | DR3+ (22) |

| | |
|---|---|
| DR4+ (31) | DR4− (10) |

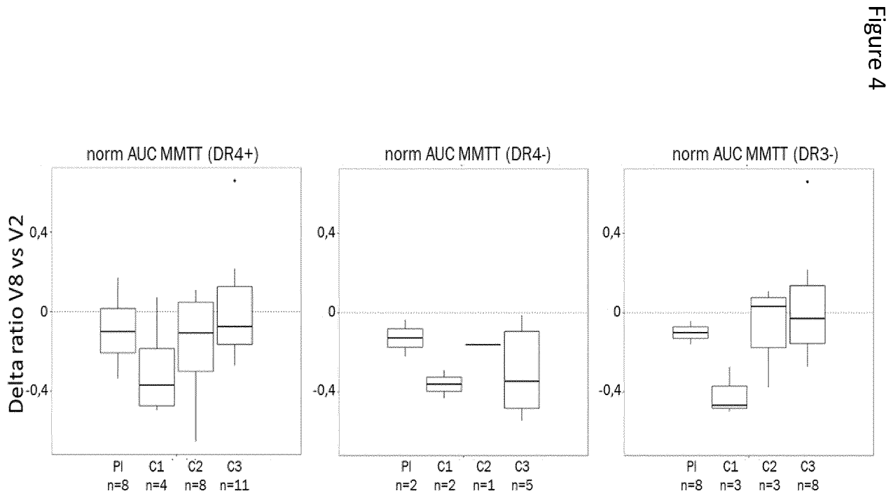

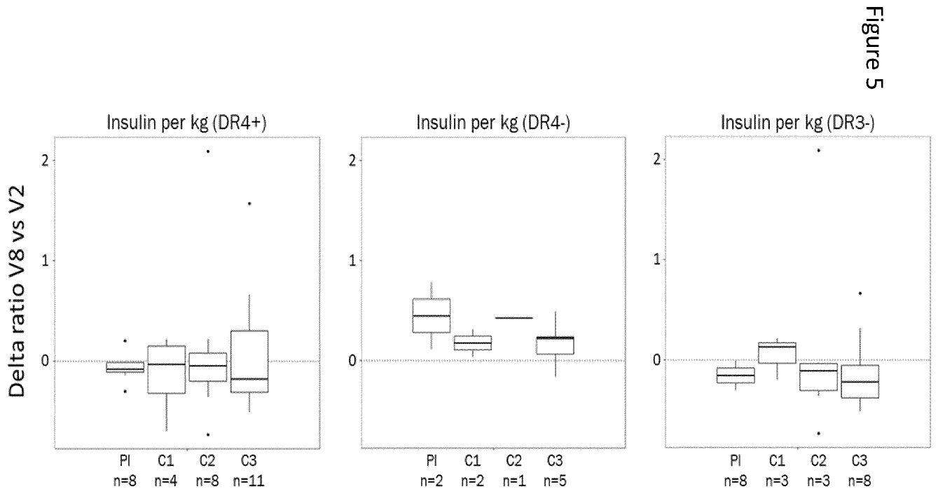

As clinical parameters examples, evolution of Area Under the Curve (AUC) C-peptide during a Mixed Meal Tolerance Test (MMTT) and daily total insulin doses per kg were investigated according to different patient's HLA-DR genotypes (FIGS. 4 and 5, respectively). Notably, patients treated with intermediate or high dose of the peptide HCPYCSLQPLALEGSLQKRG and expressing HLA-DR4 (DR4(+) or DR3(−)) showed, at 6 months (=visit 8, V8) post inclusion (=visit 2, V2), a positive trend for these two endpoints. This is not observed in populations that do not express HLA-DR4 (DR4(−)) under the test conditions of the phase Ib clinical trial.

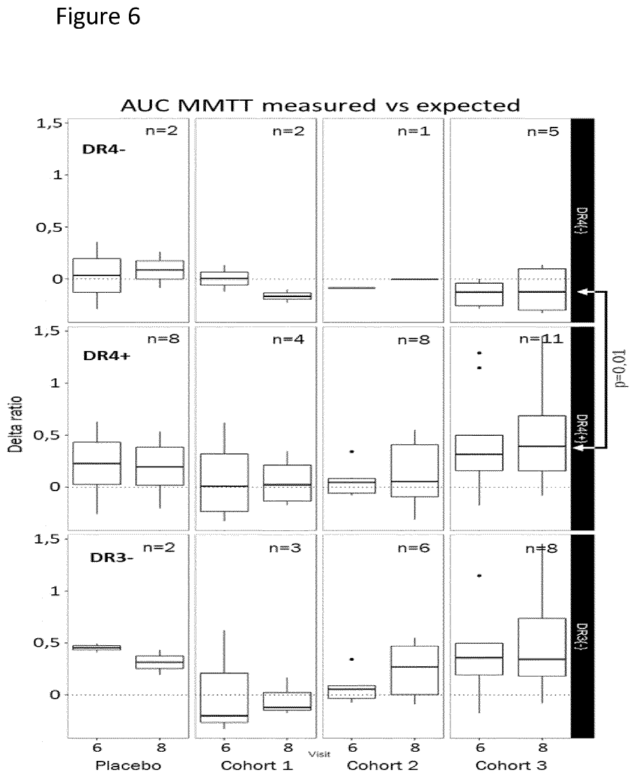

A tentative T1D disease evolution model over the first 2 years post diagnosis has been described by Greenbaum et al (Diabetes. 2012, 61(8):2066-73) based on numerous data accumulated in different clinical trials with this population of newly diagnosed patients aged 7 to 45 years old. 86% of the patients were DR3 or DR4 positive in this population. The model has used the C-peptide secretion as measured through a 2h or 4h MMTT test. We have used this model to compare the evolution of our patients with 2 goals: first, a safety aspect which has allowed to confirm that our treated patients were not showing an exacerbation of the disease (i.e. did not appear to evolve quicker than the model) and second, an efficacy aspect with the expectation to show that treated patients would evolve slower than the model. In line with the second aspect, we observed that DR4+ and DR3− subpopulations present a tendency to improvement (median decrease in C-peptide is slower than the model, delta ratio is >0) in cohort 2 and 3 at 3 months (V6) and 6 months (V8) but also in the placebo group, whereas HLA-DR4(−) subpopulations have an opposite clinical response under the test conditions of the phase Ib clinical trial (FIG. 6). Of interest, in the cohort 3 (highest dose tested), there is a significant difference in the evolution of HLA-DR4(+) and HLA-DR4(−) subpopulations. This significance is not reached in other cohorts or in the placebo group.

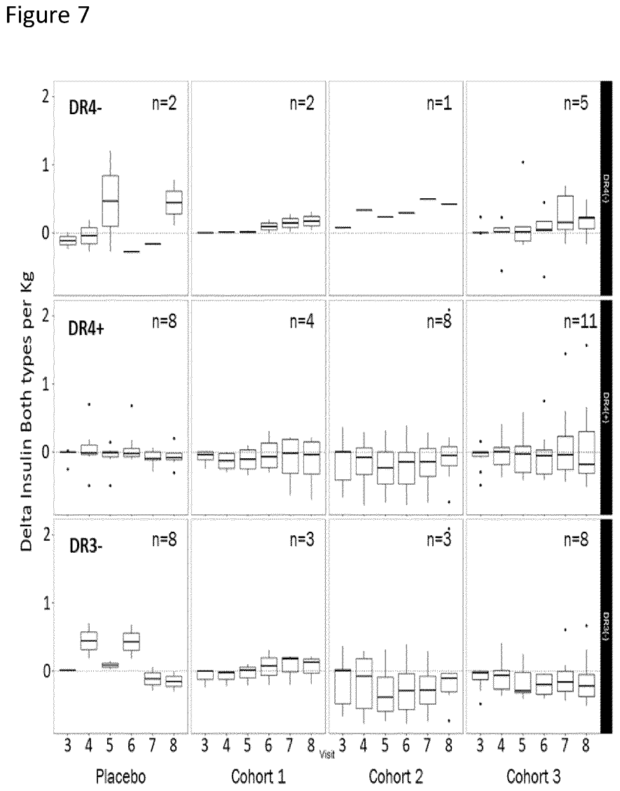

The same subgroup differences are also observed across time for the total daily insulin doses per kg. This parameter shows a tendency to decrease in cohort 2 and 3 for HLA-DR4+and DR3(−) patient's subpopulation, which is a positive response to the treatment. On the other hand, patient HLA-DR4(−) do not present this positive outcome under the test conditions of the phase Ib clinical trial (FIG. 7). For this parameter, the evolution across time in the placebo group is more heterogeneous. The efficacy of the peptide of SEQ ID NO 26 in DR3+and DR4+individuals will be further explored in larger studies with bigger sample size and stratification for HLA type.

1-4. (canceled)

5. A method of reducing the immune response to an auto-immune antigen selected from (pro)insulin or C-peptide in a patient or of treatment or prevention of type 1 diabetes in a patient, comprising administering an immunogenic peptide with a length of between 12 and 50 amino acids, comprising an oxidoreductase motif and, separated from this motif by 0 to 7 amino acids, a (pro-)insulin MHC class II T cell epitope sequence to said patient, wherein said oxidoreductase motif comprises the motif:
Zm[CST]XnC or ZmCXn[CST],
wherein n is an integer from 0 to 6,
wherein m is an integer from 0 to 2,
in which C stands for cysteine, S for serine, T for threonine, X for any amino acid and Z for any amino acid, preferably a basic amino acid,
wherein said patient has been selected based on the presence of a DR4 positive MEW class II HLA haplotype.

6-7. (canceled)

8. The method according to claim 5, wherein said (pro-)insulin MEW class II T cell epitope sequence is defined by sequence LALEGSLQK [SEQ ID NO: 3].

9. The method according to claim 5, wherein the MEW class II haplotype of said patient has been determined prior to treatment or is being determined during treatment.

10. The method according to claim 6, wherein the MHC class II haplotype of said patient has been determined using polymerase chain reaction (PCR)-based analysis, sequence analysis, electrophoretic analysis or through antibody testing.

11. The method according to claim 5, wherein patients being homozygous for HLA type DR4+ are deemed most responsive and/or wherein patients being heterozygous for HLA type DR4+ are deemed moderately responsive.

12. (canceled)

13. The method according to claim 5, wherein said peptide is administered in a dosage regimen of between 50 and 1500 μg.

14. The method according to claim 5, wherein said peptide is administered in a single dose, or in 2, 3, 4, 5, or more doses, either simultaneously or consecutively.

15. The method according to claim 5, wherein said peptide is administered through 4 bi-weekly subcutaneous or intramuscular injections according to any one of the following schemes:
1) a first subcutaneous injection with 50 μg of said peptide, followed by three consecutive subcutaneous injections of 25 μg of said peptide, each performed 2 weeks apart;
2) a first subcutaneous injection with 150 μg of said peptide, followed by three consecutive subcutaneous injections of 75 μg of said peptide, each performed 2 weeks apart; and
3) a first subcutaneous injection with 450 μg of said peptide, followed by three consecutive subcutaneous injections of 225 μg of said peptide, each performed 2 weeks apart.

16. The method according to claim 5, wherein said patients are additionally HLA-DR3 negative (HLA-DR3−).

17. The method according to claim 5, wherein said peptide is administered as a pharmaceutical composition comprising said peptide and a pharmaceutically acceptable carrier.

18. The method according to claim 5, wherein said peptide is administered as a pharmaceutical composition comprising said peptide and an adjuvant.

19-20. (canceled)

21. A method of reducing the immune response to an auto-immune antigen selected from (pro)insulin or C-peptide in a patient, comprising administering to said patient a population of cytolytic CD4+ T cells, against APC presenting insulin epitopes, obtained by a method comprising the steps of:
providing peripheral blood cells;
contacting said cells in vitro with an immunogenic peptide with a length of between 12 and 50 amino acids, comprising an oxidoreductase motif and, separated from this motif by 0 to 7 amino acids, a (pro-)insulin MHC class II T cell epitope sequence,
wherein said oxidoreductase motif comprises the motif: Zm[CST]XnC or ZmCXn[CST],
wherein n is an integer from 0 to 6,
wherein m is for an integer from 0 to 2,
in which C stands for cysteine, S for serine, T for threonine, X for any amino acid and Z for any amino acid, preferably a basic amino acid; and
expanding said cells in the presence of IL-2, wherein said patient has been selected based on the presence of a DR4 positive MHC class II HLA haplotype.

22. The method according to claim 21, wherein said patients that have been selected are additionally HLA-DR3 negative (HLA-DR3−).

23-24. (canceled)

25. The method according to claim 5, wherein said oxidoreductase motif comprises the motif: Zm[CST]XnC or ZmCXn[CST],
wherein n is an integer from 0 to 3,
wherein m stands for an integer from 0 to 2,
in which C stands for cysteine, S for serine, T for threonine, X for any amino acid and Z for a basic amino acid.

26. The method according to claim 5, wherein said oxidoreductase motif comprises the tetrapeptide sequence Cxx[CST] [SEQ ID NO: 1] or [CST]xxC [SEQ ID NO: 2].

27. (canceled)

28. The method according to claim 5, wherein said peptide comprises
a) the sequence
[SEQ ID NO: 4]
Cxx[CST]SLQPLALEGSLQK
or
[SEQ ID NO: 5]
[CST]xxCSLQPLALEGSLQK.
b the sequence
[SEQ ID NO: 6]
CxxCSLQPLALEGSLQK,
c) the sequence
[SEQ ID NO: 7]
HCxx[CST]SLQPLALEGSLQK
or
[SEQ ID NO: 8]
H[CST]xxCSLQPLALEGSLQK
or
d) the sequence
[SEQ ID NO: 9]
HCxxCSLQPLALEGSLQK.

29-31. (canceled)

32. The method according to claim 5, wherein said peptide comprises the sequence Cxx[CST] [SEQ ID NO: 1] or [CST]xxC [SEQ ID NO: 2] redox motif sequence and the sequence SLQPLALEGSLQKRG [SEQ ID NO: 20].

33. The method according to claim 5, wherein said peptide comprises or consists of amino acid sequence HCPYCSLQPLALEGSLQKRG [SEQ ID NO: 26].

34. The method according to claim 5, wherein said peptide is administered in a dosage regimen of between 100 and 1200 μg.
